(19) United States
(12) Patent Application Publication
(10) Pub. No.: US 2025/0066279 A1
(43) Pub. Date: Feb. 27, 2025

(54) PROCESS FOR PRODUCING AN ORGANIC ACID, AND CATALYST FOR SAME

(71) Applicant: MONASH UNIVERSITY, Clayton, Victoria (AU)

(72) Inventors: Akshat Tanksale, Clayton, Victoria (AU); Waqar Ahmad, Clayton, Victoria (AU); Paramita Koley, Clayton, Victoria (AU); Alan Loyd Chaffee, Clayton, Victoria (AU); Rajan Lakshman, Clayton, Victoria (AU)

(21) Appl. No.: 18/689,686

(22) PCT Filed: Sep. 7, 2022
(86) PCT No.: PCT/AU2022/051084

§ 371 Date: Mar. 6, 2024

(30) Foreign Application Priority Data
Sep. 7, 2021 (AU) 2021902895

(51) Int. Cl. C07C 51/15; B01J 20/22; B01J 37/08; C07C 51/10

(52) U.S. Cl. CPC C07C 51/15; B01J 20/226; B01J 37/08; C07C 51/10; C07C 2523/04; C07C 2523/745; C07C 2523/75; C07C 2527/20

(57) ABSTRACT

Provided herein is a process for producing a compound of formula (I), or a salt thereof: (I) catalyst. Also provided herein are catalysts which find use in the process.

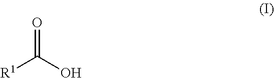

### FIELD

The present disclosure relates to processes for producing acetic acid and/or a salt thereof from carbon dioxide or carbon monoxide, and for producing formic acid and/or a salt thereof from carbon dioxide, using a metal- and carbon-containing catalyst comprising sites containing the metal in different oxidation states. The present disclosure also relates to an iron- and carbon-containing catalyst, wherein the catalyst comprises Fe (0) sites and Fe (II)/Fe (III) sites. The present disclosure also relates to a cobalt- and carbon-containing catalyst, wherein the catalyst comprises Co (0) sites and Co (II) sites.

### BACKGROUND

Fixation of overabundant atmospheric carbon dioxide is an urgent and essential research area, which may lead towards climate mitigation. Several routes for carbon dioxide conversion have been investigated, but the thermocatalytic CO₂ hydrogenation pathway is an area of focus due to its fast kinetics, and high product yields and selectivity (Li et al). Moreover, bulk chemicals such as methane, methanol, formaldehyde, dimethyl ether, gasoline-range hydrocarbons, oxymethylene dimethyl ethers and methyl formate can be produced through this process. A CO₂ based chemicals industry can not only lower the CO₂ concentration in the atmosphere but also provide revenue for offsetting capture costs.

Acetic acid is extensively used in several industrial applications, including food, chemicals, pharmaceuticals, textile, cosmetics and polymers (Pal and Nayak). It is a well-known food preservative and traditionally named as vinegar in food industry. Commercially, two major production processes are used for the synthesis of acetic acid-chemical and fermentative (Pal and Nayak; Ndoye et al). Among various chemical routes, the most common industrial processes are carbonylation of methanol (MeOH) developed by BASF, Cativa and Monsanto, in the presence of homogeneous Cobalt, Iridium and Rhodium catalysts, respectively. In the Monsanto process, acetic acid is produced from CH₃OH and fossil fuel-derived CO in the presence of CH₃I and a homogeneous rhodium-based catalyst (Qian et al; Pal and Nayak). The main reaction of acetic acid production from methanol and CO is summarized in Eq. 1.

CH₃OH+CO⇄CH₃COOH (1)

Qian et al. have reported acetic acid production via hydrocarboxylation of MeOH with carbon dioxide and hydrogen in 1,3-dimethyl-2-imidazolidinone (DMI) solvent over homogeneous Rh—Ru-based bimetallic catalyst, using a combination of LiI promoter and imidazole ligand. Imidazole played a critical role during this reaction, where it inhibited the reverse water gas shift (RWGS) reaction and promoted acetic acid production via the CO₂ hydrocarboxylation pathway (Qian et al). The same group also showed acetic acid synthesis in the presence of Rh₂(CO)₄Cl₂ homogeneous catalyst, LiCl as a co-catalyst, 4-methyl imidazole ligand and Lil as a promoter. Hasan et al. reported low yield of acetic acid (1.58 mmol/L) over NiO—C/Al₂O₃, heterogeneous catalyst at 130° C. and 35 bar total pressure of CO₂ and H₂ in 1,4 dioxane solvent after 6h of reaction. A higher amount of formic acid (FA, 4.08 mmol/L) was generated (Hasan et al).

There remains a need for further processes and catalysts which can provide means of converting carbon dioxide into useful materials, and/or which can provide access to bulk organic chemicals such as formic acid and acetic acid.

### SUMMARY OF THE INVENTION

In a first aspect, there is provided a process for producing a compound of formula (I), or a salt thereof:

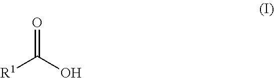

wherein R¹ is H or methyl, comprising:
a) reacting a methyl halide in the presence of hydrogen, carbon dioxide and a catalyst selected from the group consisting of i) an iron- and carbon-containing catalyst, and ii) a cobalt- and carbon-containing catalyst, to produce acetic acid and/or a salt thereof; or
b) reacting a methyl halide with carbon monoxide in the presence of a catalyst selected from the group consisting of i) an iron- and carbon-containing catalyst, and ii) a cobalt- and carbon-containing catalyst, to produce acetic acid and/or a salt thereof;
c) reacting hydrogen and carbon dioxide in the presence of a catalyst selected from the group consisting of i) an iron- and carbon-containing catalyst, and ii) a cobalt- and carbon-containing catalyst, to produce formic acid and/or a salt thereof; or
d) reacting hydrogen and carbon dioxide in the presence of a cobalt- and carbon-containing catalyst to produce acetic acid and/or a salt thereof;
wherein the iron- and carbon-containing catalyst comprises Fe (0) sites and Fe (II)/(III) sites; and wherein the cobalt- and carbon-containing catalyst comprises Co (0) sites and Co (II) sites.

In some embodiments, the process comprises:
a) reacting a methyl halide in the presence of hydrogen, carbon dioxide and an iron- and carbon-containing catalyst to produce acetic acid and/or a salt thereof; or
b) reacting a methyl halide with carbon monoxide in the presence of an iron- and carbon-containing catalyst to produce acetic acid and/or a salt thereof; or
c) reacting hydrogen and carbon dioxide in the presence of an iron- and carbon-containing catalyst, to produce formic acid and/or a salt thereof;
wherein the iron- and carbon-containing catalyst comprises Fe (0) sites and Fe (II)/(III) sites.

In some embodiments, the catalyst comprises iron-containing particles having a mean equivalent particle diameter of up to 9.8 nm, and/or a mean particle long axis of up to 9.8 nm. In some embodiments, the catalyst has more than 45 wt % iron, and has a hydrogen content in the range of from 0.4 to 0.8 wt %. In some embodiments, the catalyst has an S_{BET} value of at least 150 m² g⁻¹. In some embodiments, the catalyst is a thermally-treated iron-containing metal-organic framework (MOF), which has been thermally treated under a hydrogen-containing atmosphere. In some embodiments, the iron-containing MOF has been thermally treated under a hydrogen/argon atmosphere. In some embodiments, the iron-containing MOF has been thermally treated at a temperature in the range of from 400 to 600° C. In some embodiments, the iron-containing MOF has been thermally treated at about 500° C. In some embodiments, the catalyst comprises Fe (0) sites and Fe₃O₄ sites. In some embodiments, the wt % of iron in the catalyst is in the range of from 30 to 70 wt %. In some embodiments, the wt % of iron in the catalyst is in the range of from 45 to 55 wt %. In some embodiments, the wt % of sodium in the catalyst is in the range of from 15 to 25 wt %. In some embodiments, the wt % of carbon in the catalyst is in the range of from 10 to 20 wt %. In some embodiments, the catalyst is thermally treated MIL-88B, and wherein the MIL-88B has been thermally treated under a hydrogen/argon atmosphere at a temperature in the range of from 450 to 550° C. for a time period in the range of from 4 to 6 hours.

In some embodiments, the process comprises:
a) reacting a methyl halide in the presence of hydrogen, carbon dioxide and a cobalt- and carbon-containing catalyst to produce acetic acid and/or a salt thereof; or
b) reacting a methyl halide with carbon monoxide in the presence of a cobalt- and carbon-containing catalyst, to produce acetic acid and/or a salt thereof;
c) reacting hydrogen and carbon dioxide in the presence of a cobalt- and carbon-containing catalyst to produce formic acid and/or a salt thereof; or
d) reacting hydrogen and carbon dioxide in the presence of a cobalt- and carbon-containing catalyst to produce acetic acid and/or a salt thereof;
wherein the cobalt- and carbon-containing catalyst comprises Co (0) sites and Co (II) sites.

In some embodiments, the catalyst is a thermally-treated cobalt-containing metal-organic framework (MOF), which has been thermally treated under a hydrogen-containing atmosphere. In some embodiments, the cobalt-containing MOF has been thermally treated under a hydrogen/argon atmosphere. In some embodiments, the cobalt-containing MOF has been thermally treated at a temperature in the range of from 360 to 390° C. In some embodiments, the cobalt-containing MOF has been thermally treated at about 375° C. In some embodiments, the catalyst comprises Co (0) having a face centred cubic (FCC) structure. In some embodiments, the catalyst is thermally treated ZIF-67, and wherein the ZIF-67 has been thermally treated under a hydrogen/argon atmosphere at a temperature in the range of about 360 to 390° C. for a time period in the range of from 3 to 5 hours.

In some embodiments, the process comprises reacting a methyl halide in the presence of hydrogen, carbon dioxide and an iron- and carbon-containing catalyst to produce acetic acid and/or a salt thereof.

In some embodiments, the process comprises reacting a methyl halide with carbon monoxide in the presence of an iron- and carbon-containing catalyst to produce acetic acid and/or a salt thereof.

In some embodiments, the process comprises reacting hydrogen and carbon dioxide in the presence of a cobalt- and carbon-containing catalyst to produce acetic acid and/or a salt thereof.

In some embodiments, the process comprises reacting hydrogen and carbon dioxide in the presence of an iron- and carbon-containing catalyst, to produce formic acid and/or a salt thereof.

In some embodiments the process comprises step a) or step b), and the methyl halide is methyl iodide.

In some embodiments, the process comprises step a) and the process comprises reacting methanol with a metal halide to produce the methyl halide.

In some embodiments, the process comprises step b) and the process comprises reacting methanol with a metal halide and/or HI to produce the methyl halide.

In some embodiments, the metal halide is a metal iodide and the methyl halide is methyl iodide. In some embodiments, the metal halide is lithium iodide.

In some embodiments, the process comprises step a) and the reaction to produce acetic acid and/or a salt thereof is carried out under a pressurised hydrogen and carbon dioxide atmosphere. In some embodiments, the bar ratio of hydrogen to carbon dioxide is about 1:1. In some embodiments, the process comprises step a) and the reaction to produce acetic acid and/or a salt thereof is conducted at a pressure in the range of from 50 to 100 bar. In some embodiments, the reaction to produce acetic acid and/or a salt thereof is conducted at a pressure of about 70 bar.

In some embodiments, the process comprises step a) and the reaction to produce acetic acid and/or a salt thereof is conducted under aqueous conditions.

In some embodiments, the process comprises step a) and the reaction to produce acetic acid and/or a salt thereof is carried out with stirring and/or agitation of the reaction mixture.

In some embodiments, the process comprises step a) and the reaction to product acetic acid and/or a salt thereof comprises stirring a mixture of methanol, metal halide and water in the presence of the solid catalyst under a hydrogen and carbon dioxide atmosphere.

In some embodiments, the reaction to produce acetic acid and/or a salt thereof is carried out at a temperature in the range of from 75° C. to 200° C. In some embodiments, the reaction to produce acetic acid and/or a salt thereof is carried out at about 150° C.

In some embodiments, the process comprises step a) and the reacting step is carried out for a time period in the range of from 12 to 48 hours.

In some embodiments, process comprises step a) or step b) and, following the reaction to produce acetic acid and/or a salt thereof, the catalyst is recovered and recycled to the process.

In some embodiments, the process comprises step d) and the reacting step is carried out for a time period in the range of from 12 to 72 hours. In some embodiments, the process comprises step d), and the reaction to produce acetic acid and/or a salt thereof is conducted at a pressure in the range of from 30 to 100 bar. In some embodiments, the process comprises step d), and the reaction to produce acetic acid and/or a salt thereof is conducted at a temperature in the range of from 150 to 350° C. In some embodiments, the bar ratio of hydrogen to carbon dioxide is in the range of from 2:1 to 3:1. In some embodiments, the reaction to produce acetic acid and/or a salt thereof is carried out in the presence of a base.

In another aspect, there is provided a process for producing a product selected from the group consisting of an acetate ester, an ether acetate, a metal acetate, acetic anhydride, acrylic acid or an acrylate, comprising producing acetic acid and/or a salt thereof by a process comprising step a), step b) or step d) as defined herein, and converting the acetic acid and/or salt thereof into an acetate ester, an ether acetate, a metal acetate, acetic anhydride, acrylic acid or an acrylate.

In some embodiments, the product is selected from the group consisting of polyvinyl acetate, vinyl acetate, a metal acetate, a cellulose acetate, ethyl acetate, n-butyl acetate, isobutyl acetate, propyl acetate, ethylene glycol monoethyl ether acetate (EEA), ethylene glycol monobutyl ether acetate (EBA), and propylene glycol monomethyl ether acetate (PMA or PGMEA), acetic anhydride, acrylic acid and/or acrylate.

In another aspect, there is provided an iron- and carbon-containing catalyst, wherein the catalyst comprises Fe (0) sites and Fe (II)/Fe (III) sites, and wherein:
a) the iron particles have a mean equivalent particle diameter of up to 9.8 nm, and/or a mean particle long axis of up to 9.8 nm; and/or
b) the catalyst has more than 45 wt % iron, and has a hydrogen content in the range of from 0.4 to 0.8 wt %; and/or
c) the catalyst comprises an S_{BET} value of at least 150 m² g⁻¹; and/or
d) the catalyst is a thermally treated iron-containing metal-organic framework (MOF), which has been thermally treated under a hydrogen-containing atmosphere.

In some embodiments, at least 90% of the iron particles have an equivalent particle diameter in the range of from 3 to 20 nm, and/or at least 90% of the iron particles have a particle long axis in the range of from 3 to 20 nm. In some embodiments, the ratio of Fe (0) sites to Fe (II)/(III) sites is in the range of from 5:1 to 1:5

In some embodiments, the catalyst comprises Co (0) sites and Co (II) sites.

In some embodiments, the catalyst is a thermally-treated cobalt-containing metal-organic framework (MOF), which has been thermally treated under a hydrogen-containing atmosphere. In some embodiments, the cobalt-containing MOF has been thermally treated under a hydrogen/argon atmosphere. In some embodiments, the cobalt-containing MOF has been thermally treated at about 375° C. In some embodiments, the catalyst comprises Co (0) having a face centred cubic (FCC) structure. In some embodiments, the catalyst is thermally treated ZIF-67, and wherein the ZIF-67 has been thermally treated under a hydrogen/argon atmosphere at a temperature in the range of about 360 to 390° C. for a time period in the range of from 3 to 5 hours.

### BRIEF DESCRIPTION OF THE DRAWINGS

FIG. 1 shows PXRD patterns of the catalysts (a) Fe/CBEA, (b) T-Fe/MIL-101, and (c) T-MIL-88B, before and after catalytic tests.

FIG. 2 shows an PXRD pattern of synthesized MIL-101 and Fe/MIL-101 catalyst.

FIG. 3 shows TEM micrographs of the various studied catalysts, (a) MIL-101, (b) Fe/MIL-101, (c) T-Fe/MIL-101, (d) MIL-88B, (e) T-MIL-88B, and (f) spent T-MIL-88B after 48 h of aqueous phase CO₂ hydrogenation in the presence of CH₃OH and Lil additives; and particle size distribution of (g) T-MIL-88B, and (h) spent T-MIL-88B.

FIG. 4 shows TEM micrographs of synthesized (a) MIL-101 and (b) MIL-88B catalysts.

FIG. 5 shows a narrow scan XPS spectrum of (a) Iron 2p, (b) Chromium 2p, and (c) Carbon 1s for the studied catalysts.

FIG. 6 shows PXRD patterns for ZIF-67-T catalysts: as synthesised ZIF-67; ZIF-67-300C; ZIF-67-375C; and ZIF-67-400C.

FIG. 7 shows TEM micrographs of ZIF-67 catalysts (a) as-synthesised ZIF-67; (b) ZIF-67-375C; and (c) ZIF-67-400C.

FIG. 8 shows bar charts demonstrating the activity of various Fe-based catalysts during aqueous phase CO₂ hydrogenation in the presence of CH₃I additive at different pressures: (a) Fe/CBEA, (b) T-Fe/MIL-101, and (c) T-MIL-88B. Reaction conditions: T=150° C., H₂/CO₂=1, tR=21 h, H₂O=40 mL and stirring speed=200 RPM.

FIG. 9 shows a plot of the effect of reaction time on carboxylic acid yield and selectivity via aqueous phase CO₂ hydrogenation using T-MIL-88B in the presence of various additives, (a) CH₃I, and (b) CH₃OH and LiI. Reaction conditions: T=150° C., H₂/CO₂=1, Pₜₒₜₐₗ=70 bar at room temperature and stirring speed=200 RPM.

FIG. 10 is a bar chart showing yield and selectivity data from a recycling study of T-MIL-88B after aqueous phase CO₂ hydrogenation in the presence of CH₃OH and Lil additives. Reaction conditions: T=150° C., H₂/CO₂=1, tR=21 h, Pₜₒₜₐₗ=70 bar at room temperature and stirring speed=200 RPM.

FIG. 11 shows (a) Product yields for ZIF-67 catalysts reduced at 300° C. (▴), 375° C. (▪) and 400° C. (●) at reaction conditions: P₀=50 bar (35 bar H₂, 15 bar CO₂), T_{R}=250° C., 0.5M NaOH, 40 mL H₂O; and (b) Acetic acid selectivity of ZIF-67-T catalysts after 24h reaction time.

FIG. 12 shows (a) Product yields for ZIF-67-375C catalysts at varying initial reaction pressures (P₀) of 50 bar (▴), 60 bar (●) and 70 bar (▪) with constant P_{H2}/P_{CO2}=2.33, T_{R}=250° C., 0.5M NaOH, 40 mL H₂O; and (b) Acetic acid selectivity of ZIF-67-375C catalysts after 24h reaction time.

FIG. 13 shows a plot of acetic acid production through reaction of formic acid and CH₃I in H₂O over T-MIL-88B in the presence of hydrogen. Reaction conditions: T=150° C., n_{HCOOH}=5 mmol, n_{CH}_{₃}_{I}=10 mmol, V_{H}_{₂}_{O}=40 mL, P_{H}_{₂}=35 bar at room temperature and stirring speed=200 RPM.

FIG. 14 shows the possible reaction route for acetic acid production via aqueous phase CO₂ hydrogenation in the presence of methanol, Lil and T-MIL-88B as catalyst.

### DETAILED DESCRIPTION

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “a metal” includes mixture of two or more such metals, and the like.

The present disclosure refers to the entire contents of certain documents being incorporated herein by reference. In the event of any inconsistent teaching between the teaching of the present disclosure and the contents of those documents, the teaching of the present disclosure takes precedence.

It is to be understood that, if any prior art publication is referred to herein, such reference does not constitute an admission that the publication forms a part of the common general knowledge in the art, in Australia or any other country.

### Processes for Producing a Compound of Formula (I) or a Salt Thereof

In a first aspect, there is provided a process for producing a compound of formula (I), or a salt thereof:

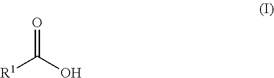

wherein R¹ is H or methyl, comprising:
a) reacting a methyl halide in the presence of hydrogen, carbon dioxide and a catalyst selected from the group consisting of i) an iron- and carbon-containing catalyst, and ii) a cobalt- and carbon-containing catalyst, to produce acetic acid and/or a salt thereof; or
b) reacting a methyl halide with carbon monoxide in the presence of a catalyst selected from the group consisting of i) an iron- and carbon-containing catalyst, and ii) a cobalt- and carbon-containing catalyst, to produce acetic acid and/or a salt thereof;
c) reacting hydrogen and carbon dioxide in the presence of a catalyst selected from the group consisting of i) an iron- and carbon-containing catalyst, and ii) a cobalt- and carbon-containing catalyst, to produce formic acid and/or a salt thereof; or
d) reacting hydrogen and carbon dioxide in the presence of a cobalt- and carbon-containing catalyst to produce acetic acid and/or a salt thereof;
wherein the iron- and carbon-containing catalyst comprises Fe (0) sites and Fe (II)/(III) sites; and wherein the cobalt- and carbon-containing catalyst comprises Co (0) sites and Co (II) sites.

The compound of formula (I) is acetic acid, formic acid, a salt of acetic acid (i.e. acetate), or a salt of formic acid (i.e. formate).

In some embodiments, the process is for producing acetic acid or a salt thereof.

In some embodiments, the process is for producing formic acid or a salt thereof.

In some embodiments, the process comprises:
a) reacting a methyl halide in the presence of hydrogen, carbon dioxide and an iron- and carbon-containing catalyst to produce acetic acid and/or a salt thereof; or
b) reacting a methyl halide with carbon monoxide in the presence of an iron- and carbon-containing catalyst to produce acetic acid and/or a salt thereof; or
c) reacting hydrogen and carbon dioxide in the presence of an iron- and carbon-containing catalyst, to produce formic acid and/or a salt thereof;
wherein the iron- and carbon-containing catalyst comprises Fe (0) sites and Fe (II)/(III) sites.

In some embodiments, the process comprises:
a) reacting a methyl halide in the presence of hydrogen, carbon dioxide and a cobalt- and carbon-containing catalyst to produce acetic acid and/or a salt thereof; or
b) reacting a methyl halide with carbon monoxide in the presence of a cobalt- and carbon-containing catalyst, to produce acetic acid and/or a salt thereof;
c) reacting hydrogen and carbon dioxide in the presence of a cobalt- and carbon-containing catalyst to produce formic acid and/or a salt thereof; or
d) reacting hydrogen and carbon dioxide in the presence of a cobalt- and carbon-containing catalyst to produce acetic acid and/or a salt thereof;
wherein the cobalt- and carbon-containing catalyst comprises Co (0) sites and Co (II) sites.

In some embodiments, the process is for producing acetic acid and/or a salt thereof, and the process comprises reacting a methyl halide in the presence of hydrogen, carbon dioxide and the iron- and carbon-containing catalyst to produce acetic acid and/or acetate.

In some embodiments, the process is for producing acetic acid or a salt thereof, and the process comprises reacting a methyl halide with carbon monoxide in the presence of the iron- and carbon-containing catalyst.

In some embodiments, the process is for producing formic acid or a salt thereof, and the process comprises reacting hydrogen and carbon dioxide in the presence of the iron- and carbon containing catalyst.

In some embodiments, the process is for producing acetic acid or a salt thereof, and the process comprises reacting hydrogen and carbon dioxide in the presence of the cobalt- and carbon-containing catalyst.

### Catalysts

In some embodiments, the processes of the present disclosure involve the use of an iron- and carbon-containing catalyst, which comprises Fe (0) sites and Fe (II)/(III) sites.

In some other embodiments, the processes of the present disclosure involve the use of a cobalt- and carbon-containing catalyst, which comprises Co (0) and Co (II) sites.

It has unexpectedly been found that catalysts containing dual iron sites (i.e. Fe (0) sites and Fe (II)/(III) sites) are effective for the production of organic acids such as acetic acid and formic acid. In particular, such catalysts can provide for good selectivity in the production of acetic acid.

Similarly, it has been found that catalysts comprising dual cobalt sites (i.e. Co (0) sites and Co (II) sites) are effective for the production of such organic acids, and again good selectivity for acetic acid production can be achieved.

For the iron- and carbon-containing catalyst, any suitable iron- and carbon-containing catalyst containing both Fe (0) and Fe (II)/(III) sites may be used. It is considered that the presence of dual types of iron sites results in a catalyst which is particularly effective in catalysing conversion of methyl halide to acetic acid.

In some embodiments, the catalyst comprises Fe (0) (e.g. metallic iron) sites and Fe₃O₄ sites.

Typically, the iron sites (e.g. Fe (0) sites and Fe (II)/(III) sites) in the catalyst have small particle size.

The sphericity of iron particles may vary. Where an iron particle is not spherical, in some embodiments the parameter equivalent particle diameter may be used to characterise the size of the particles. Equivalent particle diameter refers to the diameter of a sphere having an equivalent volume to the volume of the particle. In some other embodiments, particle long axis may be used to characterise the size of the particles. Particle long axis refers to the distance between the two furthest points on the same particle.

Any suitable technique may be used to determine particle size. For example, in some embodiments, transmission electron microscopy (TEM) is used.

In some embodiments, the catalyst comprises iron-containing particles having a mean equivalent particle diameter of up to 9.8 nm, or up to 9.7 nm, or up to 9.6 nm, or up to 9.5 nm, or up to 9.4 nm, or up to 9.3 nm, or up to 9.2 nm, or up to 9.1 nm. In some embodiments, the catalyst comprises iron-containing particles having a mean equivalent particle diameter in the range of from 8.0 to 9.8 nm, or from 8.5 to 9.7 nm, or from 8.8 to 9.7 nm, or from 9.0 to 9.7 nm, or from 9.0 to 9.5 nm, or from 9.0 to 9.3 nm. In some embodiments, the catalyst comprises iron-containing particles having a mean equivalent particle diameter of about 8.5 nm, or about 8.6 nm, or about 8.7 nm, or about 8.8 nm, or about 8.9 nm, or about 9.0 nm, or about 9.1 nm, or about 9.2 nm, or about 9.3 nm, or about 9.4 nm, or about 9.5 nm, or about 9.6 nm, or about 9.7 nm.

In some embodiments, at least 50% of the iron particles in the catalyst have an equivalent particle diameter in the range of from 1 to 20 nm, or from 2 to 18 nm, or from 4 to 16 nm. In some embodiments, at least 50% of the iron particles in the catalyst have an equivalent particle diameter in the range of from 1 to 20 nm, or from 2 to 18 nm, or from 4 to 16 nm. In some embodiments, at least 75% of the iron particles in the catalyst have an equivalent particle diameter in the range of from 1 to 20 nm, or from 2 to 18 nm, or from 4 to 16 nm. In some embodiments, at least 90% of the iron particles in the catalyst have an equivalent particle diameter in the range of from 1 to 20 nm, or from 2 to 18 nm, or from 4 to 16 nm.

In some embodiments, the catalyst comprises iron-containing particles having a mean particle long axis of up to 9.8 nm, or up to 9.7 nm, or up to 9.6 nm, or up to 9.5 nm, or up to 9.4 nm, or up to 9.3 nm, or up to 9.2 nm, or up to 9.1 nm. In some embodiments, the catalyst comprises iron-containing particles having a mean particle long axis in the range of from 8.0 to 9.8 nm, or from 8.5 to 9.7 nm, or from 8.8 to 9.7 nm, or from 9.0 to 9.7 nm, or from 9.0 to 9.5 nm, or from 9.0 to 9.3 nm. In some embodiments, the catalyst comprises iron-containing particles having a mean particle long axis of about 8.5 nm, or about 8.6 nm, or about 8.7 nm, or about 8.8 nm, or about 8.9 nm, or about 9.0 nm, or about 9.1 nm, or about 9.2 nm, or about 9.3 nm, or about 9.4 nm, or about 9.5 nm, or about 9.6 nm, or about 9.7 nm.

In some embodiments, at least 50% of the iron particles in the catalyst have a particle long axis in the range of from 1 to 20 nm, or from 2 to 18 nm, or from 4 to 16 nm. In some embodiments, at least 50% of the iron particles in the catalyst have a particle long axis in the range of from 1 to 20 nm, or from 2 to 18 nm, or from 4 to 16 nm. In some embodiments, at least 75% of the iron particles in the catalyst have a particle long axis in the range of from 1 to 20 nm, or from 2 to 18 nm, or from 4 to 16 nm. In some embodiments, at least 90% of the iron particles in the catalyst have a particle long axis in the range of from 1 to 20 nm, or from 2 to 18 nm, or from 4 to 16 nm.

The catalyst contains iron and carbon. In some embodiments, the catalyst contains more than 40 wt % iron, or more than 45 wt % iron. In some embodiments, the catalyst contains less than 55 wt % iron, or less than 50 wt % iron. In some embodiments, the catalyst contains an amount of iron in the range of from 30 to 70 wt %, or from 40 to 55 wt %, or from 45 to 55 wt %, or from 45 to 50 wt %, or from 48 to 50 wt %. In some embodiments, the catalyst contains about 49 wt % iron, or 49.3 wt % iron.

In some embodiments, the catalyst contains more than 5 wt % carbon, or more than 10 wt % carbon. In some embodiments, the catalyst contains less than 20 wt % carbon, or less than 15 wt % carbon. In some embodiments, the catalyst contains an amount of carbon in the range of from 5 to 20 wt %, or from 10 to 20 wt %, or from 10 to 15 wt %, or from 13 to 15 wt %. In some embodiments, the catalyst contains about 14 wt % carbon, or 13.7 wt % carbon.

The catalyst may contain elements other than iron and carbon. For example, it may contain hydrogen and/or sodium.

In some embodiments, the catalyst contains hydrogen. In some embodiments, the catalyst contains from 0.2 to 1.0 wt % hydrogen, or from 0.4 to 0.8 wt % hydrogen, or about 0.6 wt % hydrogen.

In some embodiments, the catalyst contains sodium. In some embodiments, the catalyst contains from 10 to 30 wt % sodium, or from 15 to 25 wt % sodium, or from 17 to 22 wt % sodium, or from 19 to 20 wt % sodium, or about 19.5 wt % sodium.

In some embodiments, the catalyst has more than 45 wt % iron, and has a hydrogen content in the range of from 0.4 to 0.8 wt %. In some embodiments, the catalyst has an iron content in the range of from 45 to 50 wt %, and a hydrogen content in the range of from 0.4 to 0.8 wt %. In some embodiments, the catalyst has an iron content in the range of from 45 to 50 wt %, and a sodium content in the range of from 17 to 22 wt %. In some embodiments, the catalyst has an iron content in the range of from 45 to 50 wt %, a hydrogen content in the range of from 0.4 to 0.8 wt %, and a sodium content in the range of from 17 to 22 wt %. In some embodiments, the catalyst has an iron content in the range of from 45 to 50 wt %, a carbon content in the range of from 10 to 15 wt %, a hydrogen content in the range of from 0.4 to 0.8 wt %, and a sodium content in the range of from 17 to 22 wt %.

In some embodiments, the catalyst has high surface area. In some embodiments, the catalyst has a specific surface area (S_{BET}) of at least 100 m² g⁻¹, at least 125 m² g⁻¹, at least 150 m² g⁻¹, or at least 160 m² g⁻¹. In some embodiments, the catalyst has a specific surface area (S_{BET}) of less than 200 m² g⁻¹, less than 180 m² g⁻¹, or less than 170 m² g⁻¹. In some embodiments, the catalyst has a specific surface area (S_{BET}) in the range of from 125 to 200 m² g⁻¹, or from 150 to 180 m² g⁻¹, or from 160 to 170 m² g⁻¹.

The catalyst may be prepared from any suitable starting materials.

In some embodiments, the catalyst is derived from an iron-containing metal-organic framework (MOF). MOFs are a group of materials containing at least two species, metal ions, and organic ligands which coordinate the metal ions and act as linkers so as to form an extended network (e.g. forming two- or three-dimensional structures). MOFs often contain significant voids. The organic units are typically mono-, di-tri- or tetravalent-ligands.

In some embodiments, the catalyst is derived from an iron-containing metal-organic framework (MOF) containing a divalent ligand. In some embodiments, the ligand is a benzene-1,4-dicarboxylic acid. In some embodiments, the ligand is 1,4-benzenedicarboxylic acid (H₂BDC).

In some embodiments, the catalyst is derived from an iron-containing metal-organic framework (MOF) which has been produced from an iron salt and a divalent ligand. In some embodiments, the catalyst is derived from an iron-containing metal-organic framework (MOF) which has been produced by admixing of i) a solution of iron nitrate in an organic solvent (e.g. dimethylformamide), and ii) a solution of 1,4-benzenedicarboxylic acid in an organic solvent (e.g. dimethylformamide), followed by addition of base (e.g. sodium hydroxide), and subsequent heating (e.g. at a temperature in the range of from 75 to 125° C.).

In some embodiments, the catalyst is derived from MIL-88B. MIL-88B is an iron-containing metal organic framework which is producible from 1,4-benzenedicarboxylic acid and iron nitrate (Liu et al, Journal of CO₂ Utilization, 2017, 21, p 100-107). The term MIL refers to Matériaux d′Institut Lavoisier.

In some embodiments, the catalyst is the product of a thermally-treated iron-containing metal-organic framework (MOF). It has been found that such catalysts are particularly effective in the production of organic acids such as acetic acid.

During thermal treatment the iron-containing metal-organic framework (MOF) is subjected to high temperature, for example at a temperature in the range of from 300 to 700° C., or in the range of from 400 to 600° C., or in the range of from 450 to 550° C., or about 500° C. The thermal treatment step may for example be conducted for a period in the range of from 2 to 12 hours, or from 3 to 9 hours, or from 4 to 6 hours, or about 5 hours.

Thermal treatment is typically carried out in the absence of oxygen. In some embodiments, the thermal treatment step is carried out under a hydrogen-containing atmosphere. In some embodiments, a mixture of hydrogen and an inert gas is used. In some embodiments, the thermal treatment step is carried out under a hydrogen/argon atmosphere. Where a hydrogen/argon atmosphere is used, the bar ratio of hydrogen to argon may for example be in the range of from 5:1 to 1:5, for example a 1:1 ratio may be used. Alternatively, where a hydrogen/argon atmosphere is used, the molar ratio of hydrogen to argon may for example be in the range of from 5:1 to 1:5, for example a 1:1 ratio may be used.

In some embodiments, the catalyst is a thermally treated iron-containing metal-organic framework (MOF) which has been subjected to thermal treatment under a hydrogen-containing atmosphere. In some embodiments, the catalyst is a thermally treated iron-containing metal-organic framework (MOF) which has been subjected to thermal treatment at a temperature in the range of from 400 to 600° C. under a hydrogen-containing atmosphere. In some embodiments, the catalyst is a thermally treated iron-containing metal-organic framework (MOF) which has been subjected to thermal treatment at a temperature in the range of from 400 to 600° C. for a period of from 4 to 6 hours under a hydrogen-containing atmosphere.

In some embodiments, the catalyst is a thermally treated iron-containing metal-organic framework (MOF) which has been subjected to thermal treatment under a hydrogen-containing atmosphere, wherein the MOF has been produced from an iron salt and a divalent ligand. In some embodiments, the catalyst is a thermally treated iron-containing metal-organic framework (MOF) which has been subjected to thermal treatment under a hydrogen-containing atmosphere, wherein the MOF has been produced from an iron salt and 1,4-benzenedicarboxylic acid (H₂BDC).

In some embodiments, the catalyst is thermally treated MIL-88B. In some embodiments, the catalyst is thermally treated MIL-88B, wherein the MIL-88B has been thermally treated under a hydrogen/argon atmosphere at a temperature in the range of from 450 to 550° C. for a time period in the range of from 4 to 6 hours.

The present disclosure also provides an iron- and carbon-containing catalyst, wherein the catalyst comprises Fe (0) sites and Fe (II)/Fe (III) sites, and wherein:
a) the iron-containing particles have a mean equivalent particle diameter of up to 9.8 nm, and/or a mean particle long axis of up to 9.8 nm; and/or
b) the catalyst has more than 45 wt % iron, and has a hydrogen content in the range of from 0.4 to 0.8 wt %; and/or
c) the catalyst comprises an S_{BET} value of at least 150 m² g⁻¹; and/or
d) the catalyst is a thermally treated iron-containing metal-organic framework (MOF), which has been thermally treated under a hydrogen-containing atmosphere.

In some embodiments, the catalyst comprises Fe (0) sites and Fe (II)/Fe (III) sites, and the iron-containing particles have a mean equivalent particle diameter of up to 9.8 nm.

In some embodiments, the catalyst comprises Fe (0) sites and Fe (II)/Fe (III) sites, and the iron-containing particles have a mean particle long axis of up to 9.8 nm.

In some embodiments, the catalyst comprises iron-containing particles having a mean equivalent particle diameter of up to 9.7 nm, or up to 9.6 nm, or up to 9.5 nm, or up to 9.4 nm, or up to 9.3 nm, or up to 9.2 nm, or up to 9.1 nm. In some embodiments, the catalyst comprises iron-containing particles having a mean equivalent particle diameter in the range of from 8.0 to 9.8 nm, or from 8.5 to 9.7 nm, or from 8.8 to 9.7 nm, or from 9.0 to 9.7 nm, or from 9.0 to 9.5 nm, or from 9.0 to 9.3 nm. In some embodiments, the catalyst comprises iron-containing particles having a mean equivalent particle diameter of about 8.5 nm, or about 8.6 nm, or about 8.7 nm, or about 8.8 nm, or about 8.9 nm, or about 9.0 nm, or about 9.1 nm, or about 9.2 nm, or about 9.3 nm, or about 9.4 nm, or about 9.5 nm, or about 9.6 nm, or about 9.7 nm.

In some embodiments, at least 50% of the iron-containing particles in the catalyst have an equivalent particle diameter in the range of from 1 to 20 nm, or from 2 to 18 nm, or from 4 to 16 nm, or from 3 to 20 nm. In some embodiments, at least 50% of the iron-containing particles in the catalyst have an equivalent particle diameter in the range of from 1 to 20 nm, or from 2 to 18 nm, or from 4 to 16 nm. In some embodiments, at least 75% of the iron-containing particles in the catalyst have an equivalent particle diameter in the range of from 1 to 20 nm, or from 2 to 18 nm, or from 4 to 16 nm, or from 3 to 20 nm. In some embodiments, at least 90% of the iron-containing particles in the catalyst have an equivalent particle diameter in the range of from 1 to 20 nm, or from 2 to 18 nm, or from 4 to 16 nm, or from 3 to 20 nm.

In some embodiments, the catalyst comprises iron-containing particles having a mean particle long axis of up to 9.7 nm, or up to 9.6 nm, or up to 9.5 nm, or up to 9.4 nm, or up to 9.3 nm, or up to 9.2 nm, or up to 9.1 nm. In some embodiments, the catalyst comprises iron-containing particles having a mean particle long axis in the range of from 8.0 to 9.8 nm, or from 8.5 to 9.7 nm, or from 8.8 to 9.7 nm, or from 9.0 to 9.7 nm, or from 9.0 to 9.5 nm, or from 9.0 to 9.3 nm. In some embodiments, the catalyst comprises iron-containing particles having a mean particle long axis of about 8.5 nm, or about 8.6 nm, or about 8.7 nm, or about 8.8 nm, or about 8.9 nm, or about 9.0 nm, or about 9.1 nm, or about 9.2 nm, or about 9.3 nm, or about 9.4 nm, or about 9.5 nm, or about 9.6 nm, or about 9.7 nm.

In some embodiments, at least 50% of the iron-containing particles in the catalyst have a particle long axis in the range of from 1 to 20 nm, or from 2 to 18 nm, or from 4 to 16 nm, or from 3 to 20 nm. In some embodiments, at least 50% of the iron-containing particles in the catalyst have a particle long axis in the range of from 1 to 20 nm, or from 2 to 18 nm, or from 4 to 16 nm. In some embodiments, at least 75% of the iron-containing particles in the catalyst have a particle long axis in the range of from 1 to 20 nm, or from 2 to 18 nm, or from 4 to 16 nm, or from 3 to 20 nm. In some embodiments, at least 90% of the iron-containing particles in the catalyst have a particle long axis in the range of from 1 to 20 nm, or from 2 to 18 nm, or from 4 to 16 nm, or from 3 to 20 nm.

In some embodiments, the catalyst comprises Fe (0) sites and Fe (II)/Fe (III) sites, and the catalyst has more than 45 wt % iron, and has a hydrogen content in the range of from 0.4 to 0.8 wt %.

In some embodiments, the catalyst contains less than 55 wt % iron, or less than 50 wt % iron. In some embodiments, the catalyst contains an amount of iron in the range of from 45 to 55 wt %, or from 45 to 50 wt %, or from 48 to 50 wt %. In some embodiments, the catalyst contains about 49 wt % iron, or 49.3 wt % iron.

In some embodiments, the catalyst contains about 0.6 wt % hydrogen.

In some embodiments, the catalyst contains more than 5 wt % carbon, or more than 10 wt % carbon. In some embodiments, the catalyst contains less than 20 wt % carbon, or less than 15 wt % carbon. In some embodiments, the catalyst contains an amount of carbon in the range of from 5 to 20 wt %, or from 10 to 20 wt %, or from 10 to 15 wt %, or from 13 to 15 wt %. In some embodiments, the catalyst contains about 14 wt % carbon, or 13.7 wt % carbon.

In some embodiments, the catalyst contains sodium. In some embodiments, the catalyst contains from 10 to 30 wt % sodium, or from 15 to 25 wt % sodium, or from 17 to 22 wt % sodium, or from 19 to 20 wt % sodium, or about 19.5 wt % sodium.

In some embodiments, the catalyst has an iron content in the range of from 45 to 50 wt %, and a hydrogen content in the range of from 0.4 to 0.8 wt %. In some embodiments, the catalyst has an iron content in the range of from 45 to 50 wt %, and a sodium content in the range of from 17 to 22 wt %. In some embodiments, the catalyst has an iron content in the range of from 45 to 50 wt %, a hydrogen content in the range of from 0.4 to 0.8 wt %, and a sodium content in the range of from 17 to 22 wt %. In some embodiments, the catalyst has an iron content in the range of from 45 to 50 wt %, a carbon content in the range of from 10 to 15 wt %, a hydrogen content in the range of from 0.4 to 0.8 wt %, and a sodium content in the range of from 17 to 22 wt %.

In some embodiments, the catalyst comprises Fe (0) sites and Fe (II)/Fe (III) sites, and comprises an S_{BET} value of at least 150 m² g⁻¹.

In some embodiments, the catalyst has a specific surface area (S_{BET}) of at least 160 m² g⁻¹. In some embodiments, the catalyst has a specific surface area (S_{BET}) of less than 200 m² g⁻¹, less than 180 m² g⁻¹, or less than 170 m² g⁻¹. In some embodiments, the catalyst has a specific surface area (S_{BET}) in the range of from 150 to 180 m² g⁻¹, or from 160 to 170 m² g⁻¹.

In some embodiments, the catalyst comprises Fe (0) sites and Fe (II)/Fe (III) sites, and the catalyst is a thermally treated iron-containing metal-organic framework (MOF), which has been thermally treated under a hydrogen-containing atmosphere.

In some embodiments, the catalyst is a metal-organic framework (MOF) which has been thermally treated under a hydrogen-containing atmosphere at a temperature in the range of from 300 to 700° C., or in the range of from 400 to 600° C., or in the range of from 450 to 550° C., or about 500° C.

In some embodiments, the catalyst is a metal-organic framework (MOF) which has been thermally treated under a hydrogen-containing atmosphere for a period in the range of from 2 to 12 hours, or from 3 to 9 hours, or from 4 to 6 hours, or about 5 hours.

In some embodiments, the catalyst is a metal-organic framework (MOF) which has been thermally treated under a hydrogen/argon atmosphere, for example under a hydrogen/argon at atmosphere at a bar ratio in the range of from 5:1 to 1:5, or about 1:1.

In some embodiments, the catalyst is a metal-organic framework (MOF) which has been thermally treated under a hydrogen/argon atmosphere at a temperature in the range of from 450 to 550° C. for a period in the range of from 4 to 6 hours.

In some embodiments, the catalyst is a metal-organic framework (MOF) which has been thermally treated under a hydrogen/argon atmosphere at a temperature in the range of from 450 to 550° C. for a period in the range of from 4 to 6 hours.

In some embodiments, the catalyst is thermally treated MIL-88B, wherein the MIL-88B has been thermally treated under a hydrogen-containing atmosphere. In some embodiments, the catalyst is thermally treated MIL-88B, wherein the MIL-88B has been thermally treated under a hydrogen/argon atmosphere at a temperature in the range of from 450 to 550° C. In some embodiments, the catalyst is thermally treated MIL-88B, wherein the MIL-88B has been thermally treated under a hydrogen/argon atmosphere at a temperature in the range of from 450 to 550° C. for a time period in the range of from 4 to 6 hours.

In the case of the cobalt- and carbon-containing catalyst, any suitable cobalt- and carbon-containing catalyst containing both Co (0) and Co (II) sites may be used in the process. It is considered that the presence of dual types of cobalt sites results in a catalyst which is particularly effective in catalysing conversion of carbon dioxide and hydrogen to acetic acid.

The cobalt- and carbon-containing catalyst used in the process comprises cobalt (0) (e.g. metallic cobalt) sites and cobalt (II) sites. The cobalt (II) sites may for example be CoO sites. The cobalt (0) sites may for example comprise Co (0) having a face centred cubic (FCC) or hexagonal close packed (HCP) structure. In some embodiments, the catalyst comprises Co (0) having a face centred cubic (FCC) structure.

In some embodiments, the catalyst used in the process is a thermally-treated cobalt-containing metal-organic framework (MOF), which has been thermally treated under a hydrogen-containing atmosphere. In some embodiments, the catalyst used in the process is a metal-organic framework (MOF) which has been thermally treated under a hydrogen-containing atmosphere for a period in the range of from 1 to 12 hours, or from 2 to 6 hours, or from 3 to 5 hours, or about 4 hours. In some embodiments, the catalyst used in the process is a cobalt-containing metal-organic framework (MOF) which has been thermally treated under a hydrogen-containing atmosphere at a temperature in the range of from 350 to 390° C., or in the range of from 370 to 380° C., or about 375° C. In some embodiments, the catalyst used in the process is a cobalt-containing metal-organic framework (MOF) which has been thermally treated under a hydrogen-containing atmosphere at a temperature in the range of from 350 to 390° C., or in the range of from 370 to 380° C., or about 375° C., for a period in the range of from 1 to 12 hours, or from 2 to 6 hours, or from 3 to 5 hours, or about 4 hours.

In some embodiments, the catalyst used in the process is a cobalt-containing metal-organic framework (MOF) which has been thermally treated under a hydrogen/argon atmosphere. In some embodiments, the catalyst used in the process is a metal-organic framework (MOF) which has been thermally treated under a hydrogen/argon atmosphere at a temperature in the range of from 350 to 390° C., or in the range of from 370 to 380° C., or about 375° C. In some embodiments, the catalyst used in the process is a metal-organic framework (MOF) which has been thermally treated under a hydrogen/argon atmosphere for a period in the range of from 1 to 12 hours, or from 2 to 6 hours, or from 3 to 5 hours, or about 4 hours. In some embodiments, the catalyst used in the process is a metal-organic framework (MOF) which has been thermally treated under a hydrogen/argon atmosphere at a temperature in the range of from 350 to 390° C., or in the range of from 370 to 380° C., or about 375° C., for a period in the range of from 1 to 12 hours, or from 2 to 6 hours, or from 3 to 5 hours, or about 4 hours.

In some embodiments, the cobalt- and carbon-containing catalyst used in the process is thermally treated ZIF-67. In some embodiments, the cobalt- and carbon-containing catalyst is thermally treated ZIF-67, wherein the ZIF-67 has been thermally treated under a hydrogen-containing atmosphere. In some embodiments, the cobalt- and carbon-containing catalyst is thermally treated ZIF-67, wherein the ZIF-67 has been thermally treated under a hydrogen/argon atmosphere. In some embodiments, the cobalt- and carbon-containing catalyst is thermally treated ZIF-67, wherein the ZIF-67 has been thermally treated under a hydrogen/argon atmosphere, at a temperature in the range from 350 to 390° C., or in the range of from 370 to 380° C., or about 375° C., for a period in the range of from 1 to 12 hours, or from 2 to 6 hours, or from 3 to 5 hours, or about 4 hours. In some embodiments, the cobalt- and carbon-containing catalyst is thermally treated ZIF-67, wherein the ZIF-67 has been thermally treated under a hydrogen/argon atmosphere at a temperature in the range of about 360 to 390° C. (e.g. about 375° C.) for a time period in the range of from 3 to 5 hours (e.g. about 4 hours).

The present disclosure also provides a cobalt- and carbon-containing catalyst which comprises Co (0) sites and Co (II) sites.

The cobalt- and carbon-containing catalyst comprises cobalt (0) (e.g. metallic cobalt) sites and cobalt (II) sites. The cobalt (II) sites may for example be CoO sites. The cobalt (0) sites may for example comprise Co (0) having a face centred cubic (FCC) or hexagonal close packed (HCP) structure. In some embodiments, the catalyst comprises Co (0) having a face centred cubic (FCC) structure.

In some embodiments, the catalyst is a thermally-treated cobalt-containing metal-organic framework (MOF), which has been thermally treated under a hydrogen-containing atmosphere. In some embodiments, the catalyst is a metal-organic framework (MOF) which has been thermally treated under a hydrogen-containing atmosphere for a period in the range of from 1 to 12 hours, or from 2 to 6 hours, or from 3 to 5 hours, or about 4 hours. In some embodiments, the catalyst is a cobalt-containing metal-organic framework (MOF) which has been thermally treated under a hydrogen-containing atmosphere at a temperature in the range of from 350 to 390° C., or in the range of from 370 to 380° C., or about 375° C. In some embodiments, the catalyst is a cobalt-containing metal-organic framework (MOF) which has been thermally treated under a hydrogen-containing atmosphere at a temperature in the range of from 350 to 390° C., or in the range of from 370 to 380° C., or about 375° C., for a period in the range of from 1 to 12 hours, or from 2 to 6 hours, or from 3 to 5 hours, or about 4 hours. In some embodiments, the catalyst is a cobalt-containing metal-organic framework (MOF) which has been thermally treated under a hydrogen/argon atmosphere. In some embodiments, the catalyst is a metal-organic framework (MOF) which has been thermally treated under a hydrogen/argon atmosphere at a temperature in the range of from 350 to 390° C., or in the range of from 370 to 380° C., or about 375° C. In some embodiments, the catalyst is a metal-organic framework (MOF) which has been thermally treated under a hydrogen/argon atmosphere for a period in the range of from 1 to 12 hours, or from 2 to 6 hours, or from 3 to 5 hours, or about 4 hours. In some embodiments, the catalyst is a metal-organic framework (MOF) which has been thermally treated under a hydrogen/argon atmosphere at a temperature in the range of from 350 to 390° C., or in the range of from 370 to 380° C., or about 375° C., for a period in the range of from 1 to 12 hours, or from 2 to 6 hours, or from 3 to 5 hours, or about 4 hours.

In some embodiments, the cobalt- and carbon-containing catalyst is thermally treated ZIF-67. In some embodiments, the cobalt- and carbon-containing catalyst is thermally treated ZIF-67, wherein the ZIF-67 has been thermally treated under a hydrogen-containing atmosphere. In some embodiments, the cobalt- and carbon-containing catalyst is thermally treated ZIF-67, wherein the ZIF-67 has been thermally treated under a hydrogen/argon atmosphere. In some embodiments, the cobalt- and carbon-containing catalyst is thermally treated ZIF-67, wherein the ZIF-67 has been thermally treated under a hydrogen/argon atmosphere, at a temperature in the range from 350 to 390° C., or in the range of from 370 to 380° C., or about 375° C., for a period in the range of from 1 to 12 hours, or from 2 to 6 hours, or from 3 to 5 hours, or about 4 hours. In some embodiments, the cobalt- and carbon-containing catalyst is thermally treated ZIF-67, wherein the ZIF-67 has been thermally treated under a hydrogen/argon atmosphere at a temperature in the range of about 360 to 390° C. (e.g. about 375° C.) for a time period in the range of from 3 to 5 hours (e.g. about 4 hours).

### Production of Acetic Acid

Where the process involves production of acetic acid or a salt thereof, the process comprises:
a) reacting a methyl halide in the presence of hydrogen, carbon dioxide and a catalyst selected from the group consisting of i) an iron- and carbon-containing catalyst, and ii) a cobalt- and carbon-containing catalyst, to produce acetic acid and/or a salt thereof; or
b) reacting a methyl halide with carbon monoxide in the presence of a catalyst selected from the group consisting of i) an iron- and carbon-containing catalyst, and ii) a cobalt- and carbon-containing catalyst, to produce acetic acid and/or a salt thereof; or
d) reacting hydrogen and carbon dioxide in the presence of a cobalt- and carbon-containing catalyst to produce acetic acid and/or a salt thereof;

In some embodiments, where the process involves production of acetic acid or a salt thereof, the process comprises reacting a methyl halide. For example, in some embodiments, the process comprises reacting a methyl halide in the presence of hydrogen, carbon dioxide and an iron- and carbon-containing catalyst to produce acetic acid and/or a salt thereof. In some other embodiments, the process comprises reacting a methyl halide with carbon monoxide in the presence of an iron- and carbon-containing catalyst to produce acetic acid and/or a salt thereof.

In some other embodiments, the process comprises reacting a methyl halide in the presence of hydrogen, carbon dioxide and a cobalt- and carbon-containing catalyst to produce acetic acid and/or a salt thereof.

In some other embodiments, the process comprises reacting a methyl halide with carbon monoxide in the presence of a cobalt- and carbon-containing catalyst to produce acetic acid and/or a salt thereof.

In some embodiments, the methyl halide is methyl iodide.

Whilst in some embodiments, the process may involve adding a methyl halide such as methyl iodide to the reactor, in some other embodiments, the methyl halide may be produced in situ before it reacts to produce acetic acid/acetate. For example, it may be produced from methanol and a metal halide (e.g. a metal iodide) or with HI, which react to produce the methyl halide (e.g. methyl iodide). Accordingly, in some embodiments, the process comprises step a) (i.e. the process comprises reacting a methyl halide in the presence of hydrogen, carbon dioxide and an iron- and carbon-containing catalyst to produce acetic acid and/or a salt thereof), and the process also comprises reacting methanol with a metal halide to produce the methyl halide.

In some embodiments, the process comprises step a) (i.e. the process comprises reacting a methyl halide in the presence of hydrogen, carbon dioxide and a cobalt- and carbon-containing catalyst to produce acetic acid and/or a salt thereof), and the process also comprises reacting methanol with a metal halide to produce the methyl halide.

Any suitable metal halide may be used. In some embodiments, the metal halide is a metal iodide, in which case the methyl halide produced is methyl iodide. Examples of metal iodides include potassium iodide and lithium iodide. In some embodiments, the metal halide is lithium iodide.

Accordingly, in some embodiments, the process comprises:
reacting methanol with a metal halide (e.g. a metal iodide such as lithium iodide) to produce methyl halide (e.g. methyl iodide); and
reacting the methyl halide in the presence of hydrogen, carbon dioxide and an iron- and carbon-containing catalyst to produce acetic acid and/or a salt thereof.

In some embodiments, the process comprises:
reacting methanol with a metal halide (e.g. a metal iodide such as lithium iodide) to produce methyl halide (e.g. methyl iodide); and
reacting the methyl halide in the presence of hydrogen, carbon dioxide and a cobalt- and carbon-containing catalyst to produce acetic acid and/or a salt thereof.

In embodiments where methanol and a metal halide (e.g. a metal iodide such as lithium iodide), the molar ratio of methanol to metal halide may for example be in the range of from 20:1 to 1:2, or from 10:1 to 1:1, or from 5:1 to 1:1, or from 3:1 to 1:1, or about 1:1.

Where the process comprises reacting a methyl halide in the presence of hydrogen, carbon dioxide and an iron- and carbon-containing catalyst, or a cobalt- and carbon-containing catalyst, the reaction to produce acetic acid and/or a salt thereof is typically carried out under pressure, e.g. under a pressurised hydrogen and carbon dioxide atmosphere. Any suitable ratio of hydrogen to carbon dioxide may be used. For example, in some embodiments the bar ratio of hydrogen to carbon dioxide is in the range of from 5:1 to 1:5, or from about 2:1 to 1:2, or about 1:1. The reaction can be carried out at a range of suitable pressures. In some embodiments, the reaction to produce acetic acid and/or a salt thereof is conducted at a pressure in the range of from 30 to 150 bar, or from 50 to 100 bar, or from 60 to 80 bar, or about 70 bar.

In some embodiments, the reaction is carried out at a hydrogen pressure in the range of from 25 to 50 bar and a carbon dioxide pressure in the range of from 25 to 50 bar, or at a hydrogen pressure in the range of from 30 to 40 bar and a carbon dioxide pressure in the range of from 30 to 40 bar, or at a hydrogen pressure of about 35 bar and a carbon dioxide pressure of about 35 bar.

Where the process comprises reacting a methyl halide in the presence of hydrogen, carbon dioxide and an iron- and carbon-containing catalyst, or a cobalt- and carbon-containing catalyst, the reaction to produce acetic acid and/or a salt thereof is typically carried out for a period of time suitable to achieve good yield of acetic acid and/or acetate. In some embodiments, the reacting step to produce acetic acid is carried out for a time period in the range of from 8 to 72 hours, or from 12 to 48 hours, or from 16 to 48 hours, or from 18 to 48 hours, or from 20 to 48 hours, or from 22 to 48 hours, or from 24 to 48 hours.

Where the process comprises reacting a methyl halide in the presence of hydrogen, carbon dioxide and an iron- and carbon-containing catalyst, or a cobalt- and carbon-containing catalyst, the reaction to produce acetic acid and/or a salt thereof may for example be carried out at a temperature in the range of from 75° C. to 200° C., or from 125° C. to 175° C., or at about 150° C.

Where the process comprises reacting a methyl halide in the presence of hydrogen, carbon dioxide and an iron- and carbon-containing catalyst, or a cobalt- and carbon-containing catalyst, the reaction to produce acetic acid and/or a salt thereof may for example be carried out at a temperature in the range of from 75° C. to 200° C., for a time period in the range of from 12 to 48 hours, and at a pressure of from 50 to 100 bar.

Where the process comprises reacting a methyl halide in the presence of hydrogen, carbon dioxide and an iron- and carbon-containing catalyst, or a cobalt- and carbon-containing catalyst, the molar: weight ratio of methyl halide to catalyst may for example be in the range of from 1 to 100 mmoles methyl halide: 0.04 to 4 g catalyst, or from 5 to 20 mmoles methyl halide: 0.2 to 0.8 g catalyst, or about 10 mmoles methyl halide to 0.4 g catalyst.

Where the process comprises reacting a methyl halide in the presence of hydrogen, carbon dioxide and an iron- and carbon-containing catalyst, or a cobalt- and carbon-containing catalyst, and the methyl halide is produced from methanol and a metal iodide, the molar: weight ratio of methanol to catalyst may for example be in the range of from 1 to 100 mmoles methanol: 0.04 to 4 g catalyst, or from 5 to 20 mmoles methanol: 0.2 to 0.8 g catalyst, or about 10 mmoles methanol to 0.4 g catalyst.

Where the process comprises reacting a methyl halide in the presence of hydrogen, carbon dioxide and an iron- and carbon-containing catalyst, or a cobalt- and carbon-containing catalyst, the reaction to produce acetic acid and/or a salt thereof may for example be conducted under aqueous conditions. For example, a mixture of water, methanol, catalyst and lithium iodide may be stirred or agitated in the presence of hydrogen and carbon dioxide. The catalyst is typically insoluble or poorly soluble in water, and so can act as a heterogeneous catalyst.

Where the process comprises reacting a methyl halide in the presence of hydrogen, carbon dioxide and an iron- and carbon-containing catalyst, or a cobalt- and carbon-containing catalyst, in the presence of water, the molar: volume ratio of methyl halide to water may for example be in the range of from 1 to 100 mmoles methyl halide: 4 to 400 ml water, or from 5 to 20 mmoles methyl halide: 20 to 80 ml water, or about 10 mmoles methyl halide to 40 ml water.

Where the process comprises reacting a methyl halide in the presence of hydrogen, carbon dioxide and an iron- and carbon-containing catalyst, or a cobalt- and carbon-containing catalyst, in the presence of water, and the methyl halide is produced from methanol and a metal iodide, the molar: weight ratio of methanol to catalyst may for example be in the range of from 1 to 100 mmoles methanol: 4 to 400 ml water, or from 5 to 20 mmoles methanol: 20 to 80 ml water, or about 10 mmoles methanol to 40 ml water.

In some embodiments, the process comprises reacting a methyl halide with carbon monoxide in the presence of an iron- and carbon-containing catalyst, or a cobalt- and carbon-containing catalyst, to produce acetic acid and/or a salt thereof. The present catalysts are considered to be useful in processes similar to the Monsanto process for producing acetic acid.

The catalyst used for such a process may for example be the same as that defined above for processes involving reaction of methyl halide in the presence of hydrogen and carbon dioxide to produce acetic acid and/or a salt thereof.

In some embodiments where the process involves production of acetic acid or a salt thereof, the process comprises reacting a methyl halide. For example, in some embodiments, the process comprises reacting a methyl halide with carbon monoxide in the presence of an iron- and carbon-containing catalyst, or a cobalt- and carbon-containing catalyst, to produce acetic acid and/or a salt thereof.

In some embodiments, the methyl halide is methyl iodide.

Whilst in some embodiments, the process may involve adding a methyl halide such as methyl iodide to the reactor, in some other embodiments, the methyl halide may be produced in situ before it reacts to produce acetic acid/acetate. For example, it may be produced from methanol and a metal halide (e.g. a metal iodide) or with HI, which react to produce the methyl halide (e.g. methyl iodide). Accordingly, in some embodiments, the process comprises step b) (i.e. the process comprises reacting a methyl halide with carbon monoxide in the presence of an iron- and carbon-containing catalyst, or a cobalt- and carbon-containing catalyst, to produce acetic acid and/or a salt thereof), and the process also comprises reacting methanol with a metal halide and/or HI to produce the methyl halide.

Any suitable metal halide may be used. In some embodiments, the metal halide is a metal iodide, in which case the methyl halide produced is methyl iodide. Examples of metal iodides include potassium iodide and lithium iodide. In some embodiments, the metal halide is lithium iodide.

Accordingly, in some embodiments, the process comprises:
reacting methanol with HI and/or a metal halide (e.g. a metal iodide such as lithium iodide) to produce methyl halide (e.g. methyl iodide); and
reacting the methyl halide with carbon monoxide in the presence of an iron- and carbon-containing catalyst, or a cobalt- and carbon-containing catalyst, to produce acetic acid and/or a salt thereof.

In embodiments where methanol and a metal halide (e.g. a metal iodide such as lithium iodide), the molar ratio of methanol to metal halide may for example be in the range of from 20:1 to 1:2, or from 10:1 to 1:1, or from 5:1 to 1:1, or from 3:1 to 1:1, or about 1:1.

Where the process comprises reacting a methyl halide with carbon monoxide in the presence of an iron- and carbon-containing catalyst, or a cobalt- and carbon-containing catalyst, the reaction to produce acetic acid and/or a salt thereof is typically carried out under pressure, e.g. under a pressurised carbon monoxide atmosphere. The reaction can be carried out at a range of suitable pressures. For example, a pressure in the range of from 30 to 100 bar may be used, or in the range of from 30 to 60 bar.

Where the process comprises reacting a methyl halide with carbon monoxide in the presence of an iron- and carbon-containing catalyst, or a cobalt- and carbon-containing catalyst, the reaction to produce acetic acid and/or a salt thereof is typically carried out for a period of time suitable to achieve good yield of acetic acid and/or acetate.

Where the process comprises reacting a methyl halide with carbon monoxide in the presence of an iron- and carbon-containing catalyst, or a cobalt- and carbon-containing catalyst, the reaction to produce acetic acid and/or a salt thereof is carried out at a suitable temperature. For example, a temperature in the range of from 150° C. to 200° C. may be used.

Where the process comprises reacting a methyl halide in the presence of hydrogen, carbon dioxide and an iron- and carbon-containing catalyst, the reaction to produce acetic acid and/or a salt thereof may for example be carried out at a temperature in the range of from 75° C. to 200° C., for a time period in the range of from 12 to 48 hours, and at a pressure of from 50 to 100 bar.

In some embodiments, the process comprises reacting hydrogen and carbon dioxide in the presence of a cobalt- and carbon-containing catalyst, to produce acetic acid and/or a salt thereof. It has been found that the use of a cobalt- and carbon-containing catalyst containing Co (0) sites and Co (II) sites, can lead to the production of acetic acid. Formic acid may also be produced as a product of the reaction.

Without being bound by any particular theory, it is considered that the reaction may involve the generation of formic acid/formate and a metal-bound methylene radical as intermediates, which react to form acetate/acetic acid.

Where the process comprises step d), the reaction to produce acetic acid and/or a salt thereof is typically carried out for a period of time suitable to achieve good yield of acetic acid and/or acetate. In some embodiments, the reacting step to produce acetic acid is carried out for a time period in the range of from 8 to 72 hours, or from 12 to 72 hours, or from 12 to 36 hours, or from 12 to 24 hours, or from 24 to 72 hours, or from 48 to 72 hours.

The reaction can be carried out at a range of suitable pressures. In some embodiments, when the process comprises step d), the reaction to produce acetic acid and/or a salt thereof is conducted at a pressure in the range of from 30 to 150 bar, or from 30 to 100 bar, or from 50 to 70 bar, or from 50 to 100 bar, or from 60 to 80 bar, or about 50 bar, or about 60 bar, or about 70 bar.

In some embodiments, when the process comprises step d), the reaction is carried out at a hydrogen pressure in the range of from 25 to 50 bar and a carbon dioxide pressure in the range of from 25 to 50 bar, or at a hydrogen pressure in the range of from 30 to 40 bar and a carbon dioxide pressure in the range of from 30 to 40 bar, or at a hydrogen pressure of about 35 bar and a carbon dioxide pressure of about 35 bar.

In some embodiments, when the process comprises step d), the bar ratio of hydrogen to carbon dioxide is in the range of from 1:1 to 5:1, or from 2:1 to 3:1, or about 2.33:1.

In some embodiments, when the process comprises step d), the reaction is carried out at a hydrogen pressure in the range of from 25 to 50 bar and a carbon dioxide pressure in the range of from 10 to 25 bar, or at a hydrogen pressure of about 35 bar and a carbon dioxide pressure of about 15 bar.

In some embodiments, when the process comprises step d), the reaction to produce acetic acid and/or a salt thereof may for example be carried out at a temperature in the range of from 150° C. to 350° C., or from 200° C. to 300° C., or from 225° C. to 275° C., or at about 250° C.

When the process comprises step d), the reaction to produce acetic acid and/or a salt thereof may for example be carried out in the presence of a suitable solvent, such as water.

In some embodiments, the process comprises step d), and the reaction is carried out using from 0.02 to 0.5 g of catalyst per mL of solvent (e.g. water), or from 0.05 to 0.2 g of catalyst per mL of solvent (e.g. water), or about 0.1 g of catalyst per mL of solvent (e.g. water).

The reaction to product acetic acid and/or a salt thereof may for example be carried out in the presence of a base, such as a metal hydroxide. Examples of metal hydroxides include sodium hydroxide, lithium hydroxide and potassium hydroxide. In some embodiments, the metal hydroxide is sodium hydroxide.

Where a base is used, it may be present at any suitable concentration to enable production of the desired product. For example, where a metal hydroxide is used (such as sodium hydroxide), it may if desired be present at a concentration in the range of from 0.1M to 1.0M, or from 0.25M to 0.75M, or about 0.5M. The processes may for example be carried out in the presence of water.

The processes to produce acetic acid or a salt thereof may be conducted using, for example, a batch, semi-continuous or continuous process.

In some embodiments, a batch process is used. For example, in some embodiments a mixture of methyl halide (or methanol and metal halide) and catalyst, and water if present, may be heated with stirring or agitation in a reactor or tank, under a pressurised atmosphere (e.g. of hydrogen and carbon dioxide, or of carbon monoxide).

In some embodiments, a semi-continuous or continuous process is used. For example, liquid and/or gaseous components may be passed over and/or through a bed of catalyst. In some embodiments a flow through reactor may be used, e.g. a fixed bed or trickle-bed reactor. A trickle-bed reactor is a reactor containing a bed of catalyst which uses downward movement of liquid components over the catalyst and either downward (co-current) or upward counter-current) movement of gaseous components (such as hydrogen and carbon dioxide, or carbon monoxide) over the catalyst.

Following production of acetic acid and/or acetate salt, the product can be recovered and/or separated from other reaction components by any suitable means.

Where the acetic acid and/or acetate is obtained as a liquid phase, the liquid phase may for example be separated from solid components by filtration or decanting. Acetic acid can be separated from other components by, for example, aqueous-organic extraction. For example, acetic acid may be extracted into a basic aqueous solution in anionic/salt form. Under acidic aqueous conditions, acetic acid may be extracted into a suitable organic solvent. Acetic acid may also be separated and/or purified by distillation, for example. In some embodiments, acetic acid is recovered by extractive distillation, or by azeotropic distillation. For example, an organic solvent such as methyl propionate may be used as an extracting solvent during distillation.

The catalyst can be recovered from the reaction by any suitable means. In some embodiments, the catalyst will be solid with most or all other reaction products (other than residual gases) being in the liquid phase. Accordingly, in some embodiments the catalyst can be recovered and separated from one or more other reaction components by filtering or decanting the product mixture. The catalyst may for example be washed with a suitable solvent, and/or dried (e.g. at elevated temperature, for example at a temperature in the range of from 40 to 100° C.).

In some embodiments, the catalyst is recovered and recycled to the process. For example, the catalyst may be recycled at least twice, or at least three times, or at least four times, or at least five times, or at least 10 times, or at least 20 times. In some embodiments, a catalyst bed may be used with other components flowing over or through the catalyst bed (for example a trickle bed reactor or fixed bed reactor may be used). In such cases, the catalyst is recycled by continued passage of reaction components over or through the catalyst bed.

### Production of Formic Acid

The present disclosure also provides a process for producing formic acid and/or a salt thereof, comprising reacting hydrogen and carbon dioxide in the presence of an iron- and carbon-containing catalyst, or a cobalt- and carbon-containing catalyst, to produce formic acid and/or a salt thereof, wherein the iron- and carbon-containing catalyst comprises Fe (0) sites and Fe (II)/(III) sites and wherein the cobalt- and carbon-containing catalyst comprises Co (0) sites and Co (II) sites.

For example, it has been found that iron- and carbon-containing catalysts comprising Fe (0) sites and Fe (II)/(III) sites can catalyse the production of formic acid from hydrogen and carbon dioxide.

As discussed above, it has also been found that the use of a cobalt- and carbon-containing catalyst containing Co (0) sites and Co (II) sites, can lead to the production of acetic acid and/or formic acid.

The catalyst used in the process may be the same as defined above for processes for producing acetic acid and/or acetate salts.

In addition, it has also been shown that iron- and carbon-containing catalysts other than those containing both Fe (0) sites and Fe (II)/(III), such as T-Fe/CBEA and T-Fe/MIL-101, can catalyse the reaction of hydrogen and carbon dioxide to produce formic acid. Accordingly, the present disclosure also comprises a process for producing formic acid and/or a salt thereof, comprising reacting hydrogen and carbon dioxide in the presence of an iron- and/or carbon-containing catalyst.

In some embodiments, the catalyst is derived from a zeolite, e.g. prepared by mixing an iron salt with a zeolite. In some embodiments, the catalyst is thermally-treated Fe/CBEA (e.g. T-Fe/CBEA).

In some embodiments, the catalyst is derived from an iron-containing metal-organic framework (MOF). In some embodiments, the catalyst is derived from an iron-containing metal-organic framework (MOF) containing a divalent ligand. In some embodiments, the ligand is a benzene-1,4-dicarboxylic acid. In some embodiments, the ligand is 1,4-benzenedicarboxylic acid (H₂BDC).

In some embodiments, the catalyst is derived from an iron-containing metal-organic framework (MOF) which has been produced from an iron salt and a divalent ligand.

In some embodiments, the catalyst is derived from an iron-containing metal-organic framework (MOF) which has been produced from a chromium salt, a divalent ligand and an iron salt.

In some embodiments, the catalyst is derived from an iron-containing metal-organic framework (MOF) which has been produced by admixing of i) a solution of iron nitrate in an organic solvent (e.g. dimethylformamide), and ii) a solution of 1,4-benzenedicarboxylic acid in an organic solvent (e.g. dimethylformamide), followed by addition of base (e.g. sodium hydroxide), and subsequent heating (e.g. at a temperature in the range of from 75 to 125° C.).

In some embodiments, the catalyst is derived from an iron-containing metal-organic framework (MOF) which has been produced by admixing of i) In some embodiments, the catalyst is derived from MIL-88B.

In some embodiments, the catalyst is derived from Fe-MIL-101.

In some embodiments, the catalyst is the product of a thermally-treated iron-containing metal-organic framework (MOF). During thermal treatment the iron-containing metal-organic framework (MOF) is subjected to high temperature, for example at a temperature in the range of from 300 to 700° C., or in the range of from 400 to 600° C., or in the range of from 450 to 550° C., or about 500° C. The thermal treatment step may for example be conducted for a period in the range of from 2 to 12 hours, or from 3 to 9 hours, or from 4 to 6 hours, or about 5 hours.

Thermal treatment is typically carried out in the absence of oxygen. In some embodiments, the thermal treatment step is carried out under a hydrogen-containing atmosphere. In some embodiments, a mixture of hydrogen and an inert gas is used. In some embodiments, the thermal treatment step is carried out under a hydrogen/argon atmosphere. Where a hydrogen/argon atmosphere is used, the bar ratio of hydrogen to argon may for example be in the range of from 5:1 to 1:5, for example a 1:1 ratio may be used.

In some embodiments, the catalyst is a thermally treated iron-containing metal-organic framework (MOF) which has been subjected to thermal treatment under a hydrogen-containing atmosphere. In some embodiments, the catalyst is a thermally treated iron-containing metal-organic framework (MOF) which has been subjected to thermal treatment at a temperature in the range of from 400 to 600° C. under a hydrogen-containing atmosphere. In some embodiments, the catalyst is a thermally treated iron-containing metal-organic framework (MOF) which has been subjected to thermal treatment at a temperature in the range of from 400 to 600° C. for a period of from 4 to 6 hours under a hydrogen-containing atmosphere.

In some embodiments, the catalyst is a thermally treated iron-containing metal-organic framework (MOF) which has been subjected to thermal treatment under a hydrogen-containing atmosphere, wherein the MOF has been produced from an iron salt and a divalent ligand. In some embodiments, the catalyst is a thermally treated iron-containing metal-organic framework (MOF) which has been subjected to thermal treatment under a hydrogen-containing atmosphere, wherein the MOF has been produced from an iron salt, a divalent ligand and a chromium salt. In some embodiments, the catalyst is a thermally treated iron-containing metal-organic framework (MOF) which has been subjected to thermal treatment under a hydrogen-containing atmosphere, wherein the MOF has been produced from an iron salt and 1,4-benzenedicarboxylic acid (H₂BDC). In some embodiments, the catalyst is a thermally treated iron-containing metal-organic framework (MOF) which has been subjected to thermal treatment under a hydrogen-containing atmosphere, wherein the MOF has been produced from an iron salt, 1,4-benzenedicarboxylic acid (H₂BDC) and a chromium salt.

In some embodiments, the catalyst is thermally treated MIL-88B. In some embodiments, the catalyst is thermally treated MIL-88B, wherein the MIL-88B has been thermally treated under a hydrogen/argon atmosphere at a temperature in the range of from 450 to 550° C. for a time period in the range of from 4 to 6 hours.

In some embodiments, the catalyst is thermally treated Fe/MIL-101 (T-Fe/MIL-101). In some embodiments, the catalyst is thermally treated Fe/MIL-101 (T-Fe/MIL-101), wherein the Fe/MIL-101 has been thermally treated under a hydrogen/argon atmosphere at a temperature in the range of from 450 to 550° C. for a time period in the range of from 4 to 6 hours.

Where the process comprises reacting hydrogen and carbon dioxide in the presence of a catalyst, the reaction to produce formic acid and/or a salt thereof is typically carried out under pressure, e.g. under a pressurised hydrogen and carbon dioxide atmosphere. Any suitable ratio of hydrogen to carbon dioxide may be used. For example, in some embodiments the bar ratio of hydrogen to carbon dioxide is in the range of from 5:1 to 1:5, or from about 2:1 to 1:2, or from 1:1 to 5:1, or from 2:1 to 3:1 or about 2.33:1 or about 1:1. The reaction can be carried out at a range of suitable pressures. In some embodiments, the reaction to produce formic acid and/or a salt thereof is conducted at a pressure in the range of from 30 to 150 bar, or from 30 to 100 bar, or from 50 to 70 bar, or from 50 to 100 bar, or from 60 to 80 bar, or about 70 bar. In some embodiments, the reaction is carried out at a hydrogen pressure in the range of from 25 to 50 bar and a carbon dioxide pressure in the range of from 25 to 50 bar, or at a hydrogen pressure in the range of from 30 to 40 bar and a carbon dioxide pressure in the range of from 30 to 40 bar, or at a hydrogen pressure of about 35 bar and a carbon dioxide pressure of about 35 bar.

In some embodiments, the reaction is carried out at a hydrogen pressure in the range of from 25 to 50 bar and a carbon dioxide pressure in the range of from 10 to 25 bar, or at a hydrogen pressure of about 35 bar and a carbon dioxide pressure of about 15 bar.

Where the process comprises reacting hydrogen and carbon dioxide in the presence of a catalyst, the reaction to produce formic acid and/or a salt thereof is typically carried out for a period of time suitable to achieve good yield of formic acid and/or acetate. Optimal reaction time may depend on the catalyst used. In some embodiments, where the catalyse comprises Fe (0) sites and Fe (II)/(III) sites, the reacting step to produce formic acid may be carried out for a time period in the range of from 3 to 12 hours, or from 6 to 10 hours, or about 8 hours. In some embodiments, where the catalyst comprises Co (0) sites and Co (II) sites, the reacting step to produce formic acid may be carried out for a time period in the range of from 3 to 12 hours, or from 3 to 6 hours, or about 3 hours.

Where the process comprises reacting hydrogen and carbon dioxide in the presence of a catalyst, the reaction to produce formic acid and/or a salt thereof may for example be carried out at a temperature in the range of from 75° C. to 350° C., or from 75° C. to 200° C., or from 125° C. to 175° C., or at about 150° C., or from 200° C. to 300° C., or from 225° C. to 275° C., or about 250° C.

The reaction to produce formic acid and/or a salt thereof may for example be carried out in the presence of water.

The reaction to product formic acid and/or a salt thereof may for example be carried out in the presence of a base, such as a metal hydroxide. Examples of metal hydroxides include sodium hydroxide, lithium hydroxide and potassium hydroxide. In some embodiments, the metal hydroxide is sodium hydroxide.

Where a base is used, it may be present at any suitable concentration to enable production of the desired product. For example, where a metal hydroxide is used (such as sodium hydroxide), it may if desired be present at a concentration in the range of from 0.1M to 1.0M, or from 0.25M to 0.75M, or about 0.5M.

The process to produce formic acid or a salt thereof may be conducted using, for example, a batch, semi-continuous or continuous process.

In some embodiments, a batch process is used.

In some embodiments, a semi-continuous or continuous process is used. For example, liquid and/or gaseous components may be passed over and/or through a bed of catalyst. In some embodiments a flow through reactor may be used, e.g. a fixed bed or trickle-bed reactor. A trickle-bed reactor is a reactor containing a bed of catalyst which uses downward movement of liquid components over the catalyst and either downward (co-current) or upward counter-current) movement of gaseous components (such as hydrogen and carbon dioxide) over the catalyst.

Following production of formic acid and/or formate salt, the product can be recovered and/or separated from other reaction components by any suitable means.

Where the formic acid and/or formate is obtained as a liquid phase, the liquid phase may for example be separated from solid components by filtration or decanting.

Formic acid may also be separated and/or purified by distillation, for example.

The catalyst can be recovered from the reaction by any suitable means. In some embodiments, the catalyst will be solid with most or all other reaction products (other than residual gases) being in the liquid phase. Accordingly, in some embodiments the catalyst can be recovered and separated from one or more other reaction components by filtering or decanting the product mixture. The catalyst may for example be washed with a suitable solvent, and/or dried (e.g. at elevated temperature, for example at a temperature in the range of from 40 to 100° C.).

In some embodiments, the catalyst is recovered and recycled to the process. For example, the catalyst may be recycled at least twice, or at least three times, or at least four times, or at least five times, or at least 10 times, or at least 20 times. In some embodiments, a catalyst bed may be used with other components flowing over or through the catalyst bed (for example a trickle bed reactor or fixed bed reactor may be used). In such cases, the catalyst is recycled by continued passage of reaction components over or through the catalyst bed.

### Downstream Products

The organic acids and salts thereof produced by the process, e.g. the acetic acid and/or a salt thereof produced by the process, can also be used to prepare other useful products. Accordingly, also provided herein is a process for producing a product selected from the group consisting of an acetate ester, an ether acetate, a metal acetate, acetic anhydride, acrylic acid or an acrylate, comprising producing acetic acid and/or a salt thereof by a process comprising step a) or step b) as defined herein, and converting the acetic acid and/or salt thereof into an acetate ester, an ether acetate, a metal acetate, acetic anhydride, acrylic acid or an acrylate.

In some embodiments, the product is selected from the group consisting of polyvinyl acetate, vinyl acetate, a metal acetate, a cellulose acetate, ethyl acetate, n-butyl acetate, isobutyl acetate, propyl acetate, ethylene glycol monoethyl ether acetate (EEA), ethylene glycol monobutyl ether acetate (EBA), and propylene glycol monomethyl ether acetate (PMA or PGMEA), acetic anhydride, acrylic acid and/or acrylate.

Products such as metal acetates can be produced by, for example, treating acetic acid with a metal hydroxide. Acetate esters such as ethyl acetate, n-butyl acetate, isobutyl acetate, propyl acetate, ethylene glycol monoethyl ether acetate (EEA), ethylene glycol monobutyl ether acetate (EBA), and propylene glycol monomethyl ether acetate (PMA or PGMEA), may for example be produced by reaction of acetic acid with the appropriate alcohol, for example by heating in the presence of acid, and with removal of water.

As a further example, the desired alcohol may be reacted with acetic anhydride, which may itself be generated from acetic acid.

Cellulose acetates may be produced, for example, by reaction of cellulose with acetic anhydride and acetic acid in the presence of a further acid such as sulfuric acid, e.g. to produce cellulose triacetate. If desired, selective hydrolysis of the cellulose triacetate may be used to produce cellulose having the desired level of esterification.

Vinyl acetate may for example be prepared by vapour phase reaction of acetic acid with ethylene in the presence of oxygen and an appropriate catalyst, e.g. a palladium catalyst, for example at a temperature in the range of from 175 to 200° C. and a pressure of from about 5 to 10 bar.

Acetic anhydride can be produced by, for example the Tennessee Eastman acetic acid process, involving conversion of methyl acetate (itself preparable from acetic acid) to methyl iodide and an acetate salt. Carbonylation of methyl iodide provides acetyl iodide which reacts with acetic acid or acetate salt to provide acetic anhydride.

The present disclosure is also illustrated by the following numbered clauses:
1 A process for producing a compound of formula (I), or a salt thereof:

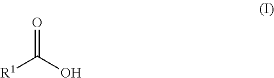

wherein R¹ is H or methyl, comprising:
   a) reacting a methyl halide in the presence of hydrogen, carbon dioxide and an iron- and carbon-containing catalyst to produce acetic acid and/or a salt thereof; or
   b) reacting a methyl halide with carbon monoxide in the presence of an iron- and carbon-containing catalyst to produce acetic acid and/or a salt thereof; or
   c) reacting hydrogen and carbon dioxide in the presence of an iron- and carbon-containing catalyst, to produce formic acid and/or a salt thereof;
wherein the iron- and carbon-containing catalyst comprises Fe (0) sites and Fe (II)/(III) sites.
2. The process according to clause 1, wherein the catalyst comprises iron-containing particles having a mean equivalent particle diameter of up to 9.8 nm, and/or a mean particle long axis of up to 9.8 nm.
3. The process according to clause 1 or 2, wherein the catalyst has more than 45 wt % iron, and has a hydrogen content in the range of from 0.4 to 0.8 wt %.
4. The process according to any of clauses 1 to 3, wherein the catalyst has an S_{BET} value of at least 150 m² g⁻¹.
5. The process according to any of clauses 1 to 4, wherein the catalyst is a thermally-treated iron-containing metal-organic framework (MOF), which has been thermally treated under a hydrogen-containing atmosphere.
6 The process according to clause 5, wherein the iron-containing MOF has been thermally treated under a hydrogen/argon atmosphere.
7. The process according to clause 5 or 6, wherein the iron-containing MOF has been thermally treated at a temperature in the range of from 400 to 600° C.
8 The process according to any of clauses 5 to 7, wherein the iron-containing MOF has been thermally treated at about 500° C.
9. The process according to any of clauses 1 to 8, wherein the catalyst comprises Fe (0) sites and Fe₃O₄ sites.
10. The process according to any of clauses 1 to 9, wherein the wt % of iron in the catalyst is in the range of from 30 to 70 wt %.
11. The process according to clause 10, wherein the wt % of iron in the catalyst is in the range of from 45 to 55 wt %.
12. The process according to any of clauses 1 to 11, wherein the wt % of sodium in the catalyst is in the range of from 15 to 25 wt %.
13. The process according to any of clauses 1 to 12, wherein the wt % of carbon in the catalyst is in the range of from 10 to 20 wt %.
14. The process according to any of clauses 1 to 13, wherein the catalyst is thermally treated MIL-88B, and wherein the MIL-88B has been thermally treated under a hydrogen/argon atmosphere at a temperature in the range of from 450 to 550° C. for a time period in the range of from 4 to 6 hours.
15. The process according to any of clauses 1 to 14, wherein the process comprises reacting a methyl halide in the presence of hydrogen, carbon dioxide and an iron- and carbon-containing catalyst to produce acetic acid and/or a salt thereof.
16. The process according to any of clauses 1 to 14, wherein the process comprises reacting a methyl halide with carbon monoxide in the presence of an iron- and carbon-containing catalyst to produce acetic acid and/or a salt thereof.
17. The process according to any of clauses 1 to 14, wherein the process comprises reacting hydrogen and carbon dioxide in the presence of an iron- and carbon-containing catalyst, to produce formic acid and/or a salt thereof.
18. The process according to any of clauses 1 to 16, wherein the process comprises step a) or step b), and the methyl halide is methyl iodide.
19. The process according to any of clauses 1 to 15 or 18, wherein the process comprises step a) and the process comprises reacting methanol with a metal halide to produce the methyl halide.
20. The process according to any of clauses 1 to 14, 16 or 18, wherein the process comprises step b) and the process comprises reacting methanol with a metal halide and/or HI to produce the methyl halide.
21. The process according to clause 19 or 20, wherein the metal halide is a metal iodide and the methyl halide is methyl iodide.
22. The process according to clause 21, wherein the metal halide is lithium iodide.
23. The process according to any of clauses 1 to 15, 18 to 19 or 21 to 22, wherein the process comprises step a) and the reaction to produce acetic acid and/or a salt thereof is carried out under a pressurised hydrogen and carbon dioxide atmosphere.
24. The process according to clause 23, wherein the bar ratio of hydrogen to carbon dioxide is about 1:1.
25. The process according to any of clauses 1 to 15, 18 to 19, or 21 to 24, wherein the process comprises step a) and the reaction to produce acetic acid and/or a salt thereof is conducted at a pressure in the range of from 50 to 100 bar.
26. The process according to clause 25, wherein the reaction to produce acetic acid and/or a salt thereof is conducted at a pressure of about 70 bar.
27. The process according to any of clauses 1 to 15, 18 to 19, or 21 to 26, wherein the process comprises step a) and the reaction to produce acetic acid and/or a salt thereof is conducted under aqueous conditions.
28. The process according to any of clauses 1 to 15, 18 to 19, or 21 to 27, wherein the process comprises step a) and the reaction to produce acetic acid and/or a salt thereof is carried out with stirring and/or agitation of the reaction mixture.
29. The process according to any of clauses 19 or 21 to 28, wherein the process comprises step a) and the reaction to product acetic acid and/or a salt thereof comprises stirring a mixture of methanol, metal halide and water in the presence of the solid catalyst under a hydrogen and carbon dioxide atmosphere.
30. The process according to any of clauses 1 to 16 or 18 to 29, wherein the reaction to produce acetic acid and/or a salt thereof is carried out at a temperature in the range of from 75° C. to 200° C.
31. The process according to clause 30, wherein the reaction to produce acetic acid and/or a salt thereof is carried out at about 150° C.
32. The process according to any of clauses 1 to 15, 18 to 19, or 21 to 31, wherein the process comprises step a) and the reacting step is carried out for a time period in the range of from 12 to 48 hours.
33. The process according to any of clauses 1 to 16 or 18 to 32, wherein the process comprises step a) or step b) and, following the reaction to produce acetic acid and/or a salt thereof, the catalyst is recovered and recycled to the process.
34. A process for producing a product selected from the group consisting of an acetate ester, an ether acetate, a metal acetate, acetic anhydride, acrylic acid or an acrylate, comprising producing acetic acid and/or a salt thereof by a process comprising step a) or step b) as defined in any of clauses 1 to 16 or 18 to 33, and converting the acetic acid and/or salt thereof into an acetate ester, an ether acetate, a metal acetate, acetic anhydride, acrylic acid or an acrylate.
35. The process according to clause 34, wherein the product is selected from the group consisting of polyvinyl acetate, vinyl acetate, a metal acetate, a cellulose acetate, ethyl acetate, n-butyl acetate, isobutyl acetate, propyl acetate, ethylene glycol monoethyl ether acetate (EEA), ethylene glycol monobutyl ether acetate (EBA), and propylene glycol monomethyl ether acetate (PMA or PGMEA), acetic anhydride, acrylic acid and/or acrylate.
36. An iron- and carbon-containing catalyst, wherein the catalyst comprises Fe (0) sites and Fe (II)/Fe (III) sites, and wherein:
a) the iron particles have a mean equivalent particle diameter of up to 9.8 nm, and/or a mean particle long axis of up to 9.8 nm; and/or
b) the catalyst has more than 45 wt % iron, and has a hydrogen content in the range of from 0.4 to 0.8 wt %; and/or
c) the catalyst comprises an S_{BET} value of at least 150 m² g⁻¹; and/or
d) the catalyst is a thermally treated iron-containing metal-organic framework (MOF), which has been thermally treated under a hydrogen-containing atmosphere.
37. The iron- and carbon-containing catalyst according to clause 36, wherein at least 90% of the iron particles have an equivalent particle diameter in the range of from 3 to 20 nm, and/or at least 90% of the iron particles have a particle long axis in the range of from 3 to 20 nm.
38. The iron- and carbon-containing catalyst according to clause 36 or 37, wherein the ratio of Fe (0) sites to Fe (II)/(III) sites is in the range of from 5:1 to 1:5.

### Examples

The present disclosure is further illustrated by the following non-limiting examples.

### 1. Materials

Iodomethane (CH₃I, 99.5%), formic acid (HCOOH, ≥95%), lithium Iodide (LiI, 99.9%), terephthalic acid (H₂BDC, 98%), chromium chloride hexahydrate (CrC13.6H₂O, 98%), and iron nitrate nonahydrate (Fe(NO₃)₃. 9H₂O, 98%) were purchased from the Sigma Aldrich. Commercial zeolite-beta (CBEA, SiO₂/Al₂O₃=38) was received from Zeolyst International. Methanol (HPLC grade) was obtained from the Scharlau Chemicals. Milli-Q water was used for catalysts synthesis (MIL-101 and Fe/CBEA) and acetic acid production experiments.

### 2. Catalyst Synthesis

### 2.1 Preparation of Fe/CBEA

A wet impregnation process was used for the Fe/CBEA synthesis as described in the literature (Ahmad et al, 2020). The loading of Fe was fixed as 10 wt % in this catalyst. Typically, Fe(NO₃) 3.9H₂O (7.2343 g) was dissolved in Milli-Q water (30 mL) by using 100 mL Schott bottle and stirred for 15 min at 65° C. to prepare a homogeneous mixture of Fe solution. Thereafter, 9 g of CBEA support was immersed in this solution under stirring and maintained for 6 h at the same temperature to achieve an even dispersion of Fe particles on CBEA support. The mixture was dried in oven at 100° C. followed by calcination at 550° C. with a heating rate of 5° C./min for 5 h in muffle furnace. The synthesised catalyst was reduced in an environment of H₂/Ar (1:1 v/v) gas mixture at 400° C. for 5 h with heating rate of 5° C./min prior to the carbon dioxide conversion experiment.

### 2.2 Preparation of Fe/MIL-101 and Thermal Decomposition to Produce T-Fe/MIL-101

10 mmol of H₂BDC and 10 mmol CrCl₃.6H₂O were poured into a Teflon-lined autoclave. Subsequently, Milli-Q water (72 ml) was added to it. The reaction mixture was sonicated for 30 minutes followed by stirring for another 30 minutes at 500 rpm. Thereafter, the autoclave was kept in the oven at 205° C. for 24 h and allowed to cool to room temperature. The resulting solid suspension was transferred into centrifuge tube. Initially, the centrifugation was performed at 1000 rpm for 3-4 min to remove the unreacted H₂BDC present in the reaction mixture. Thereafter, the centrifugation was carried out at 5000 rpm for 10 minutes. The solid sample was then washed with DMF three times and then dried in an oven at 70° C. for 12 h. The synthesised material was named as MIL-101.

For the Fe/MIL-101 synthesis, 2.7 g of MIL-101 was suspended in 70 mL ethanol in a Schott bottle and sonicated for 30 minutes. Separately, 2.17 g Fe(NO₃) 3.9H₂O was dissolved in 20 mL ethanol in a different Schott bottle and stirred for 15 minutes. The latter solution was poured into the former suspension of MIL-101 in ethanol. Then the Schott bottle, which contained Fe(NO₃) 3 solution, was washed with 10 mL ethanol three times and poured into the MIL-101 suspension to ensure complete transfer of the Fe precursor. The resultant mixture was sonicated for 30 minutes followed by stirring at 50° C. at 500 rpm for 5-6 h. Finally, the resulting reaction mixture was dried in an oven at 80 C for 2-3 days. The synthesized catalyst was renamed as Fe/MIL-101. The Fe loading was fixed as 10 wt % in the synthesized catalyst. Prior to catalytic activity tests, this catalyst was thermally degraded under 100 mL/min H₂/Ar (1:1) gas mixture at 500° C. for 5 h with a heating rate of 5° C./min and allowed to cool naturally in 50 mL/min Ar atmosphere and denoted as T-Fe/MIL-101.

### 2.3 Preparation of MIL-88B and Thermal Decomposition to Produce T-MIL-88B

A hydrothermal method was adopted during the synthesis of MIL-88B as described in the literature (Liu et al). In a typical procedure, 12.12 g of Fe salt (Fe(NO₃) 3.9H₂O) was dissolved in 75 ml DMF under stirring (500 RPM) in a Schott bottle. Separately, H₂BDC (4.98 g) and DMF (75 ml) were added in a 250 mL Teflon-liner under stirring (500 RPM). Both Fe and H₂BDC solutions were stirred further for 15 min at room temperature. The Fe solution was then poured into H₂BDC precursor solution. Moreover, 12 mL NaOH solution (4.0 M) was slowly transferred into Fe and H₂BDC solution mixture and stirred again for 30 min at room temperature. The Teflon-liner was subsequently placed into autoclave and sealed. It was then put inside an oven for 24 h at 100° C. After cooling to room temperature, MIL-88B particles were collected from this mixture via centrifugation at 7000 RPM for 10 min and washed three times with DMF and methanol, respectively. Finally, the as synthesized MIL-88B was dried overnight in the oven at 80° C. and denoted as MIL-88B. Thermolysis of MIL-88B (2 g) was conducted at 500° C. for 5 h with a ramp of 5° C./min in 100 mL/min H₂/Ar (1:1) environment, cooled to room temperature under Ar at 50 mL/min atmosphere and denoted as T-MIL-88B.

### 2.4 Preparation of ZIF-67 and Thermal Decomposition to Produce ZIF-67T

ZIF-67 was synthesised via an aqueous phase reaction at ambient conditions, as has been previously reported (Potter et al. Chen et al.). Some modification to these reported methods was made to scale up the yield as per the following.

2.25 g of cobalt nitrate hexahydrate and 27.5 g of 2-methylimidazole was separately dissolved in 100 mL and 15 mL of Milli-Q water, respectively. The solution containing cobalt nitrate hexahydrate was then slowly added to the 2-methylimidazole solution in a dropwise manner and stirred continuously for 6 h at room temperature. Purple precipitates were formed and collected by centrifugation at 10,000 rpm for 10 minutes. The sample was purified by washing with Milli-Q water (3 times) and then with methanol (3 times) to remove any unreacted species. Finally, the sample was dried at 80° C. overnight.

The as-synthesised ZIF-67 was reduced under 25% H₂/Ar (100 ml/min) atmosphere, with a ramp rate of 2° C./min and held at the desired reduction temperature (T) for 4h to produce the thermally transformed catalyst denoted as ZIF-67-T. After reduction, the catalyst was naturally cooled and kept under argon flow (50 mL/min) overnight. ZIF-67 was reduced at 3 different temperatures to produce thermally transformed catalysts labelled ZIF-67-300° C., ZIF-67-375° C. and ZIF-67-400° C.

### 3. Catalyst Characterisation

### 3.1 Methodology (Fe/MIL-101 and MIL-88B)

The crystal structure of studied materials was investigated with Powder X-ray diffraction (PXRD) by using a Rigaku MiniFlex device. The powder catalysts were loaded in a zero-background sample holder and scanned between 2-80° 20 with 4°/min scan speed at 15 mA and 40 kV.

Nitrogen sorption analysis was conducted with Micromeritics 3Flex 3500 machine to find the type of adsorption isotherm, Brunauer-Emmett-Teller (BET) surface area and Barrett-Joyner-Halenda (BJH) pore distribution. Tecani T20 machine was used to capture the transmission electron microscopy (TEM) images of the catalysts. All samples were dispersed in ethanol and immobilised onto the surface of a holy carbon grid followed by drying in air prior to analysis.

ThermoScientific K-Alpha machine was utilized for X-ray photoelectron spectroscopy (XPS) at 1486.6 eV Ephoton and coupled with monochromatic Al Kα radiations. The binding energy (B.E.) baseline correction was conducted by adjusting the C 1 s peaks at 284.8 eV. For thermally decomposed samples, the pyrolysis was performed ex-situ prior to XPS characterization of these samples. Shimadzu DTG-60H thermogravimetric analyser was used to check the thermal stability of Fe/MIL-101 and MIL-88B. Both samples were analysed in the temperature range of 100-800° C. with a ramp of 5° C./min under Ar atmosphere.

### 3.2 Methodology (ZIF-67)

The crystal structure of studied materials was investigated with Powder X-ray diffraction (PXRD) by using a Rigaku Miniflex instrument with Cuka radiation ( )=0.154 nm), operated at 40 kV and 15 mA. Samples were loaded onto a zero-background holder and scanned over 2-80° (20) at a speed of 4°/min. The diffraction patterns were used to investigate the crystallinity of the catalysts and for phase identification of the cobalt species.

Catalysts were also characterised by transmission electron microscopy (TEM) using the Tecnai T20 microscope to determine the size and dispersion of metal nanoparticles. Powdered samples were dispersed in ethanol and sonicated prior to being dropped onto a holy carbon grid for imaging.

### 3.2 Results

### 3.2.1 Powdered X-ray Crystallography (PXRD) of Fe-catalysts

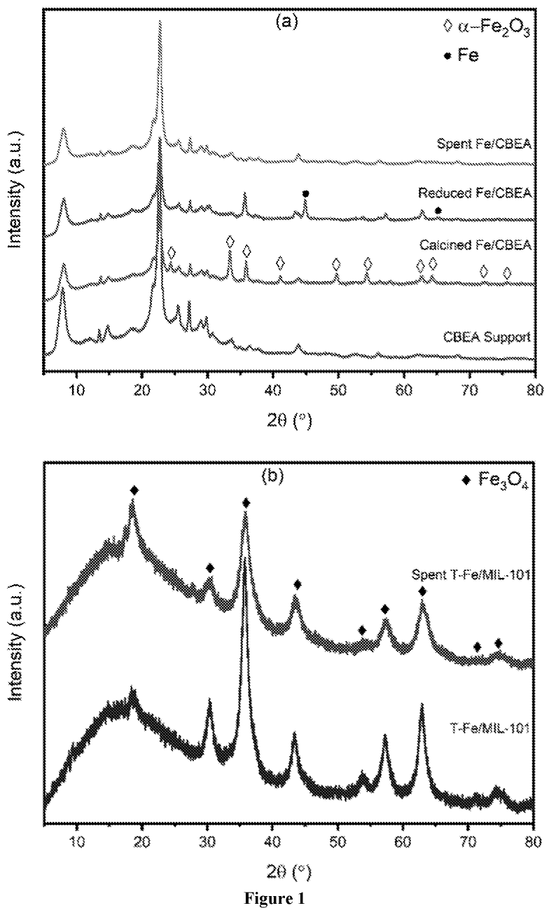

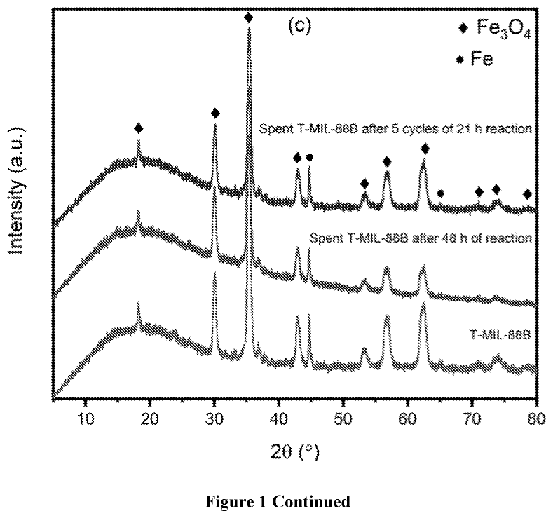

The calcined Fe/CBEA catalyst showed characteristic peaks of α-Fe₂O₃, most of which were not observed in the reduced catalyst. Instead, the reduced catalyst showed Fe⁰ peaks at 20=44.7° and 65° and residual α-Fe₂O₃ peaks at 35.98° and 62.83°. However, there were no Fe° or α-Fe₂O₃ peaks detected in the spent catalyst which indicated leaching of Fe from the catalyst support (FIG. 1a).

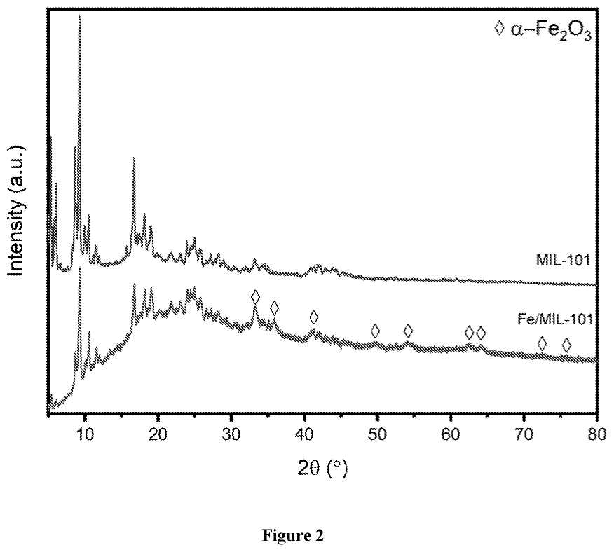

Both the fresh and the spent T-Fe/MIL-101 catalyst showed peaks corresponding to Fe₃O₄, but no Fe⁰ peaks (FIG. 1b), suggesting that the catalyst was stable after the reaction. Whereas, α-Fe₂O₃ peaks were observed in Fe/MIL-101 as shown in FIG. 2.

Finally, the T-MIL-88B catalyst showed peaks corresponding to Fe₃O₄ and Fe° (FIG. 1c), which remained steady after a single run of 48 h reaction time in the presence of CH₃I and 5 cycles of 21 h reaction time each, in the presence of Lil and CH₃OH. Only Fe₃O₄ peaks have been reported after the pyrolysis of MIL-88B at 500° C. for 4 h under nitrogen atmosphere (Liu et al). However, both Fe₃O₄ and Fe⁰ peaks were present for T-MIL-88B in this study, which may be due to the reducing hydrogen atmosphere during the thermal decomposition of MIL-88B.

### 3.2.2 Transmission Electron Microscopy (TEM)

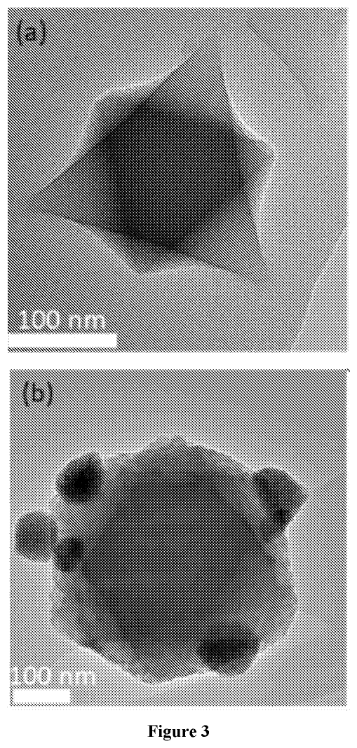

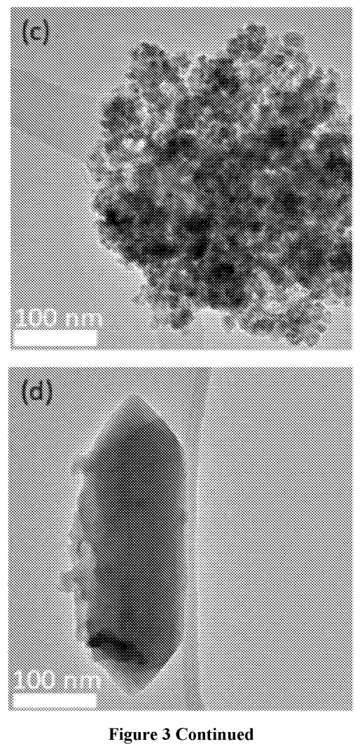

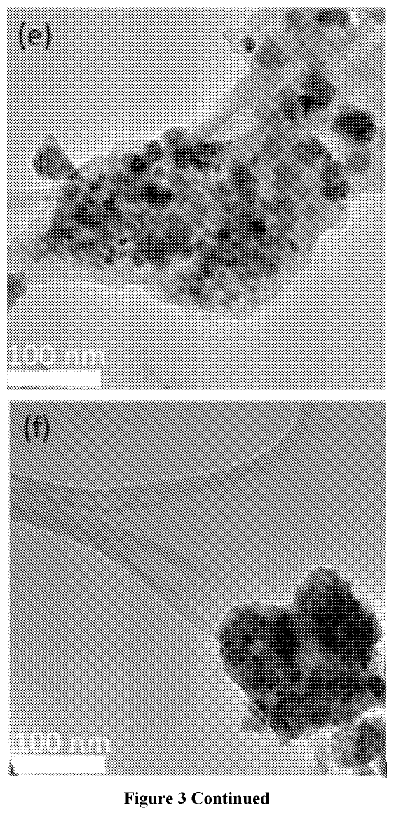

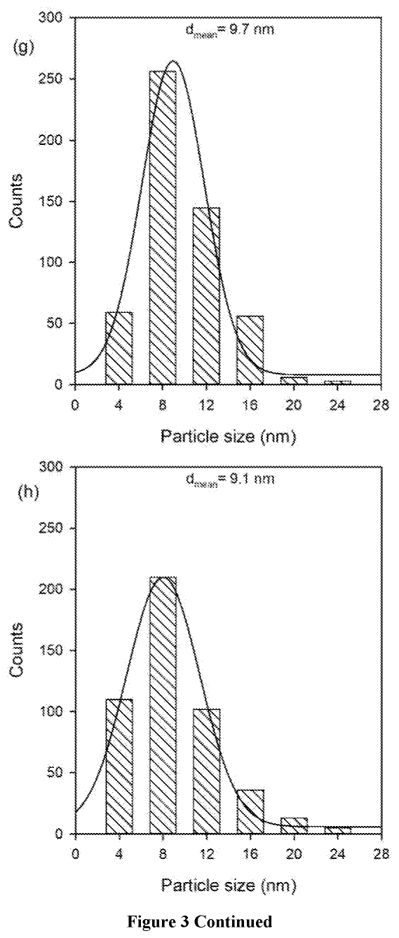

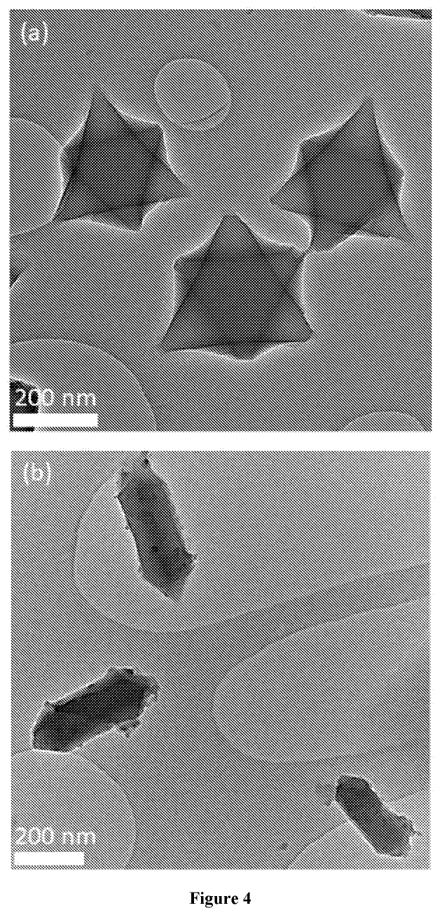

FIG. 3 a-f shows the TEM images of MIL-101, Fe/MIL-101, T-Fe/MIL-101, MIL-88B, T-MIL-88B, and spent T-MIL-88B, respectively. MIL-101 shows an octahedral shape of ca. 200-300 nm size (FIG. 3a and FIG. 4a) which matches with published works (Zhao et al). Wet impregnated Fe nanoparticles on MIL-101 (Fe/MIL-101) were approximately 50-100 nm in size (FIG. 3b), whereas after thermal decomposition, T-Fe/MIL-101 exhibited approximately 5-30 nm particles (FIG. 3c). FIG. 3d and FIG. 4b show the characteristic fusiform rod-shaped morphology of MIL-88B with ˜360 nm length and 90 nm width similar to published works (Liu et al). After thermal decomposition, T-MIL-88B shows a narrow range of Fe°/Fe₃O₄ nanoparticle which are well-dispersed over the carbonaceous support (FIG. 3e). FIG. 3f shows that the catalyst retains its structure after 48h of reaction. FIG. 3 g-h illustrates the particle size distribution (PSD) for T-MIL-88B and spent T-MIL-88B, respectively. 525 and 476 particles were measured from multiple images which showed most of the particles in 4-16 nm for both fresh and spent T-MIL-88B, respectively. The peaks were observed at 8 nm with average particle sizes of 9.7 and 9.1 nm for fresh and spent T-MIL-88B, respectively which suggested that the studied catalyst is stable and indicates reusability in the process.

### 3.2.3 X-Ray Photoelectron Spectroscopy (XPS)

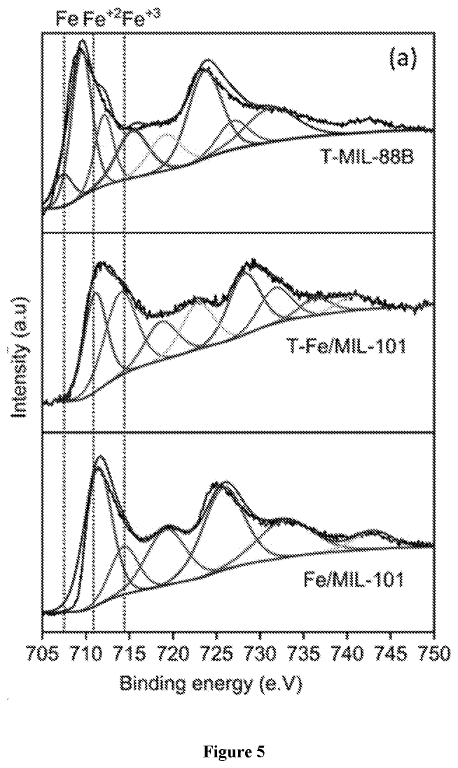

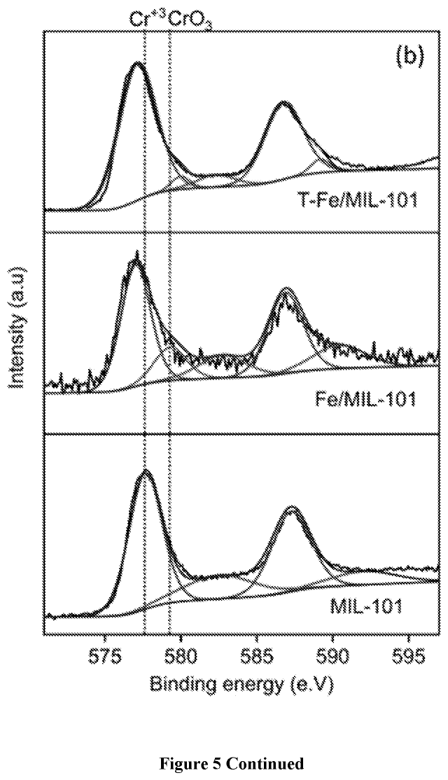

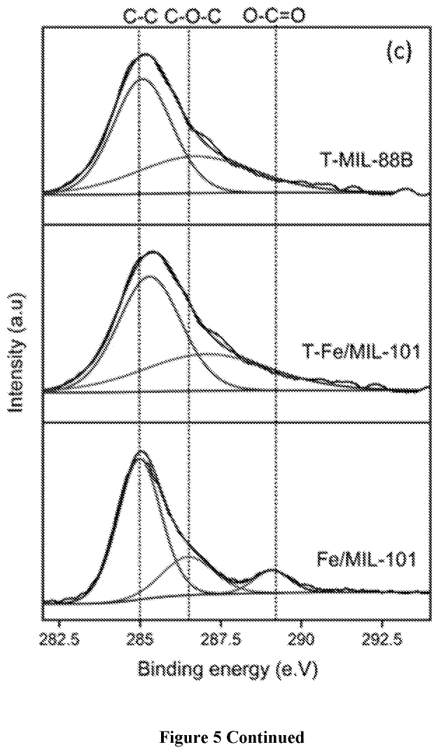

The surface chemical oxidation state of the element in the different catalysts was evaluated by X-Ray photoelectron spectroscopy (XPS) study and represented in FIG. 5. For T-MIL-88B (FIG. 5a), Fe 2p_{3/2} XPS spectrum exhibited three peaks, including a peak at 706.9 eV corresponding to metallic iron (Ma et al). Moreover, the other two peaks at 710.1 and 712.3 eV which are correlated to Fe⁺² and Fe⁺³ oxidation state of iron and the satellite peaks for these aforementioned oxidation state appeared at 716.6 and 719.8 eV (Koley et al). In the Fe 2p region of T-MIL-88B, Fe2p_{1/2} and Fe2p_{3/2} peaks are situated 710.1 and 723.8 eV, where, the spin orbital splitting is 13.7 eV that indicated the presence of Fe₃O₄ in T-MIL-88B (Lee et al). The XPS spectra of Fe 2p_{3/2} in T-Fe/MIL-101 exhibited two peaks at 711.7 and 712.4 eV which is related to Fe⁺² and Fe⁺³ along with two satellite peaks at 718.1 and 722.4 eV. Furthermore, Fe2p_{1/2} and Fe2p_{3/2} of Fe⁺² appeared at 711.7 and 725.4 eV and the spin orbital splitting is 13.7 eV which interpreted the existence of Fe₃O₄ in T-Fe/MIL-101. Metallic Fe peak is absent in this catalyst which is in good agreement with PXRD results. For Fe/MIL-101 catalyst, Fe 2p_{3/2} XPS spectra also contained both Fe⁺² and Fe-3 at 711.6 and 713.4 eV, respectively. However, the spin orbital splitting for Fe2p_{1/2} and Fe2p_{3/2} is 14.1 eV (711.7 and 725.8 eV) which suggested the absence of Fe₃O₄ phase.

FIG. 5b represents the Cr XPS spectra of MIL-101, Fe/MIL-101 and T-Fe/MIL-101 catalysts. In MIL-101, Cr 2p XPS spectra contained only one peak at 577.6 eV which is corresponds to Cr⁺³ oxidation state (Lee et al). For Fe/MIL-101, Cr XPS spectra attributed to two peaks at 577.2 and 578.8 eV which are mainly resembles with Cr⁺³ and CrO₃ (Guo et al). The negative binding energy shift (0.4 eV) of Cr⁺³ as compared to Cr⁺³ present in MIL-101 is most likely due to the interfacial electronic interaction (charge transfer) between Cr and Fe after the inclusion of Fe in MIL-101 (Koley et al). The Cr spectra for T-Fe/MIL-101, Cr XPS spectra mainly consisted with Cr” 3 peak at 577.1 eV and the amount of CrO₃ is very less as compared to Fe/MIL-101 which may be due to the thermal decomposition of Fe/MIL-101 under hydrogen atmosphere that reduces the oxidised Cr species on catalyst surface.

The C Is XPS spectra for Fe/MIL-101 (FIG. 5c) shows three different types of C peak at 285, 286.4 and 288.5 which belongs to C—C, C—O—C and O—C═O (Fee-rong et al). The C Is XPS spectra of both T-Fe/MIL-101 and T-MIL-88B contains only two peaks corresponding to C—C and C—O—C, whereas, the O—C═O peak is absent, which may be due to the thermal decomposition of both Fe/MIL-101 and MIL-88B under hydrogen atmosphere reducing the oxygen content in the catalyst.

### 3.2.4 Thermogravimetric Analysis (TGA)

A thermogravimetric analysis of Fe/MIL-101 and MIL-88B was carried out (results not shown). For Fe/MIL-101, a weight loss in the range of 50-250° C. was observed, due to the evaporation of water and removal of free terephthalates inside the pore of MOF. Thereafter, the observed main weight loss in the temperature range of 270 to 670° C. is due to the degradation of organic ligand in the framework of MOF which is attributed to the collapse of the framework. The weight loss of MIL-88B before 250° C. corresponds to the removal of water and excess DMF from the framework. For MIL-88B, weight loss occurs in the temperature ranges of 300 to 500° C., due to the degradation of H₂BDC and the breakdown of the framework. An observed step in the TGA profile of between 550-650° C. is most likely due to the carbonization of the framework and the formation of Fe₃O₄-carbon composites.

### 3.2.5 Elemental Analysis of T-MIL-88

The elemental composition of T-MIL-88B is described in Table 1 which demonstrates 49.3 wt % of Fe contents in the synthesised catalyst.

TABLE 1
Elemental composition of thermally
transformed MIL-88B (T-MIL-88B)

| Elements | Weight percentages (%) |
|---|---|
| Fe* | 49.3 |
| Na* | 19.5 |
| C** | 13.7 |
| H** | 0.6 |
| N** | 0.0 |
| S** | 0.0 |
| O*** | 16.9 |

*measured by XRF analysis
**measured by CHNS analysis
***calculated on the basis of difference

### 3.2.6 BET of Studied Catalysts

The nitrogen linear isotherms of different iron based CBEA, MIL-101 and MIL-88B catalysts were measured (results not shown). The nitrogen sorption of CBEA and Fe/CBEA catalyst represent the combination of type I and IV isotherm which interpreted that the catalyst mostly consisted of micropores. The two steps nitrogen isotherm indicated the presence of both micropores and mesopores in the catalyst. The micropores filling mainly occurred in the ranges of 0<p/p°<0.05 and the mesoporous filling which leads to the appearance of hysteresis loop (H₃ type) was observed in the ranges of 0.4<p/p°<0.9. The surface area of CBEA and reduced Fe/CBEA are 673.9 and 390.1 m²/g, respectively. The surface area of reduced Fe/CBEA is lower than the CBEA support itself which is most likely the blockage of the pore with iron. The BJH pore size distribution of CBEA support (not shown) revealed the presence of mesopores in between the ranges of 1.9-50 nm. The pore diameter and the mesopore volume of this support (CBEA) is 5.3 nm and 0.11 cm³/g, respectively which has been changed minorly after the impregnation of iron as shown in Table 2. Whereas, the micropore volume of the CBEA support has changed significantly from 0.21 to 0.12 cm³/g in reduced Fe/CBEA catalyst.

The N₂ adsorption-desorption isotherm of MIL-101 displayed typical type I isotherm along with the secondary uptakes at p/p° ˜ 0.1 and p/p° ˜ 0.2 which is good agreement with the literature (Férey et al). Fe. Fe/MIL-101 also exhibits the similar type of isotherm as compared to MIL-101. After impregnation of Fe in MIL-101, the surface area of the catalyst decreased from 2473.7 cm²/g to 1831.3 cm²/g which is maybe due to the blocking of the pores with metal that is enforced by the BJH results of these two catalysts. The micropores and mesopores volume of Fe/MIL-101 are 0.09 and 0.51 cm³/g respectively which are comparatively lower than the micropore (0.35 cm³/g) and mesopore (0.60 cm³/g) volume of MIL-101 due to pore blockage. The total pore volume of Fe/MIL-101 is also lower than MIL-101 (Table 2). The degraded MIL-101 (T-Fe/MIL-101) catalyst shows (figure not shown) type II isotherm with H3 hysteresis loop which has a good resemblance with the results obtained for MIL-101 (Cr)/RGO/ZnFe204 nanocomposite. The surface area of T-Fe/MIL-101 is very lower (73.5 m²/g) as compared to MIL-101 and Fe/MIL-101 which has a good resemblance with the findings obtained by Farisabadi et al. The poor surface area of this aforementioned catalyst is most likely due to the collapse of organic framework which was previously articulated by Li et al. for MIL-101-Cr MOF that was thermally treated at 550° C. (Li et al). In the low-pressure region, the absence of adsorption suggested that all micropores in MIL-101 are collapsed and the existence of hysteresis loop in the high-pressure region indicated that presence of mesopores.

N₂ adsorption-desorption isotherms of MIL-88B and T-MIL-88B were measured (figure not shown). MIL-88B exhibited the type I isotherm that suggesting the microporous MOF with high surface area (410.6 m²/g) which is almost similar with the results reported by Vu et al. The average pore diameter is 6.1 nm with total pore volume (both micropore and mesopore) of 0.32 cm³/g. The N₂ adsorption-desorption isotherm of T-MIL-88B is type II with H2 (b)-type hysteresis loop. Due to the presence of mesopores, the high-pressure region of the isotherm consisted with hysteresis loop. Table 2 shows that the surface area of T-MIL-88B catalyst is lower (160.6 m²/g) than MIL-88B which is most likely due to collapse of organic matrix.

TABLE 2
Physical and textural characteristics
of studied supports and catalysts

| | ^{†}S_{BET} | ^{††}D_{Pore} | ^{├}V_{micro} | ^{├├}Vₘₑₛₒ | ^{├├├}Vₜₒₜₐₗ |
|---|---|---|---|---|---|
| Nanomaterials | (m²/g) | (nm) | (cm³/g) | (cm³/g) | (cm³/g) |
| CBEA | 673.9 | 5.3 | 0.21 | 0.11 | 0.32 |
| Reduced 10% Fe/CBEA | 390.1 | 6.4 | 0.12 | 0.13 | 0.25 |
| MIL-101 | 2473.7 | 3.0 | 0.35 | 0.60 | 0.95 |
| 10% Fe/MIL-101 | 1831.3 | 2.8 | 0.09 | 0.51 | 0.60 |
| T-10% Fe/MIL-101 | 73.5 | 20.2 | 0.00 | 0.38 | 0.38 |
| MIL-88B | 410.6 | 6.1 | 0.09 | 0.23 | 0.32 |
| T-MIL-88B | 160.6 | 14.2 | 0.04 | 0.27 | 0.31 |

^{†}surface area via Brunauer-Emmett-Teller (BET) method.
^{††}Mean desorption diameter of pore via Barrett-Joyner-Halenda (BJH) method.
^{├}Cumulative desorption pore volume via Barrett-Joyner-Halenda (BJH) method.
^{├├}Micropore volume via t-plot.
^{├├├}Total volume of pores.

### 3.2.7 Powdered X-Ray Crystallography (PXRD) of Co-Catalysts

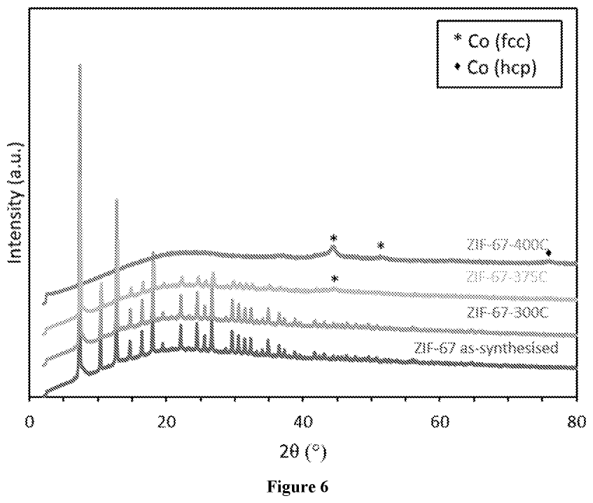

XRD patterns for the synthesised ZIF-67-T catalyst are shown in FIG. 6. Reduction at 300° C. did not result in degradation of the characteristic peaks seen in the as-synthesised ZIF-67 and cobalt ions were not found to be reduced to the metallic cobalt phase. Increasing the reduction temperature to 375° C. resulted in lower intensities for the characteristic ZIF-67 peaks indicating some minor loss in crystallinity and cobalt ions started to get reduced as the fcc phase of metallic cobalt was detected. Further increasing the degradation temperature to 400° C. removed all of the characteristic ZIF-67 peaks indicating complete loss of the overall crystalline structure and both the fcc and hcp phases of metallic cobalt were observed.

### 3.2.8 Transmission Electron Microscopy (TEM) of Co-catalysts

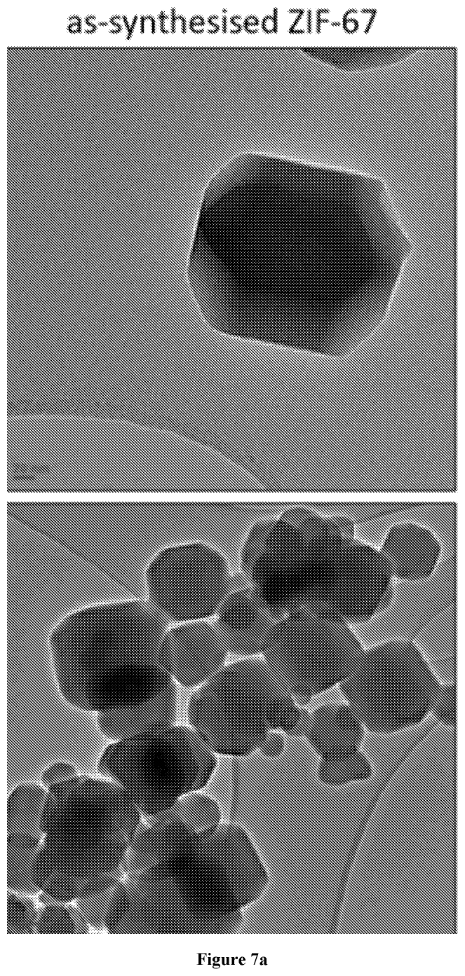

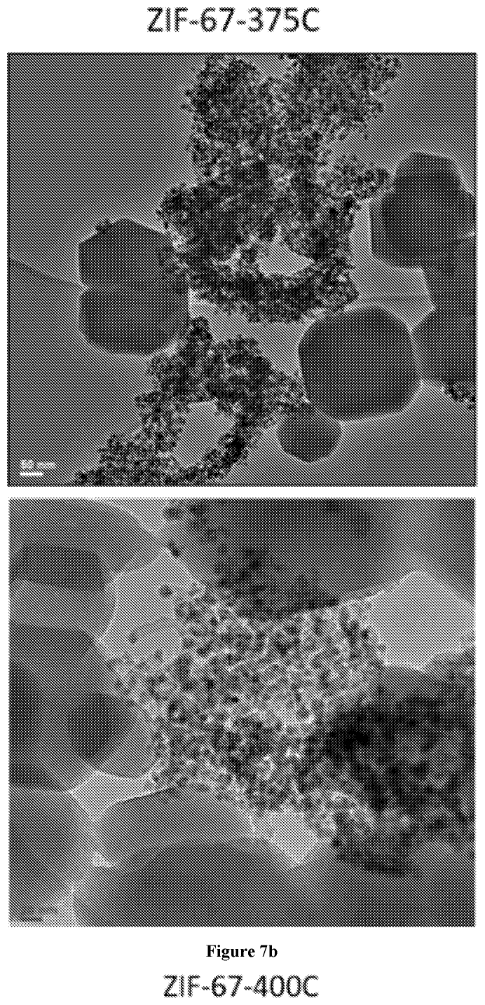

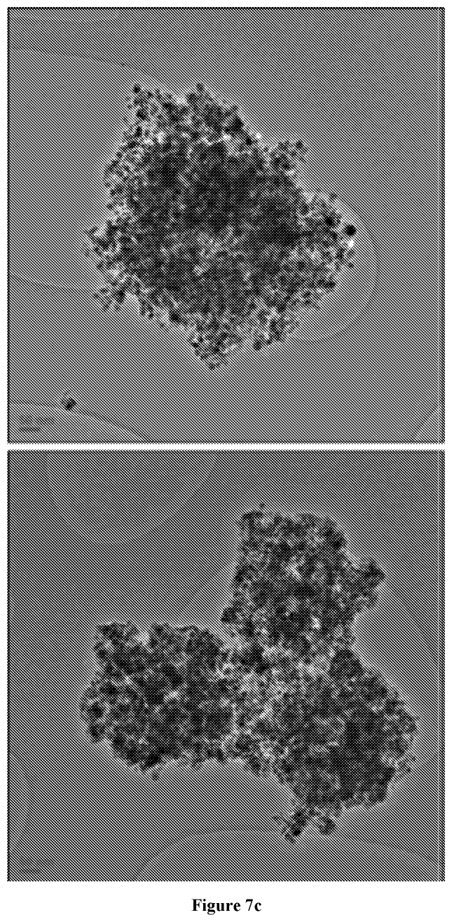

TEM analysis of the thermally transformed ZIF-67 catalysts showed a clear effect of reduction temperature on the structural morphology and dispersion of cobalt nanoparticles as seen in FIGS. 7a-c. TEM micrographs for ZIF-67-375C showed that some level of crystal structure remained as some particles began to thermally transform. ZIF-67-400C particles, however, were more amorphous which supported the XRD results showing a loss of crystallinity.

### 4. Aqueous Phase CO₂ Conversion

4.1 Aqueous Phase CO₂ Conversion using Fe-catalysts

All aqueous phase CO₂ conversion experiments were performed in a 100 mL Teflon-lined autoclave batch reactor. Typically, 0.4 g of thermally decomposed catalyst (T-MIL-88B) and 40 mL water was added to the reactor and CH₃I (10 mmol) was carefully poured into it and sealed. The reactor was purged with hydrogen in triplicate to eliminate the presence of air. The reactor was then pressurised with CO₂ up to 35 bar, followed by H2 up to a total pressure of 70 bar at room temperature to achieve CO₂:H2 ratio of 1:1. The reactor was heated to 150° C. under continuous stirring at 200 RPM for 21 h. After 21 h of reaction, the reactor was allowed to cool to room temperature and the remaining gases were carefully vented from it before dissembling. The catalyst was recovered from the liquid product mixture by centrifugation at 8500 RPM for 1 h. The same procedure was repeated for different total pressures at equimolar CO₂:H₂ ratio and different catalysts (T-Fe/MIL-101 and Fe/CBEA). The liquid sample was analysed at intervals for the best catalyst to check the extent of reaction against time at 150° C., equimolar CO₂:H₂ under 70 bar with 200 RPM stirring speed. The liquid samples were analysed using an HPLC (Agilent 1220 Infinity) equipped with a C18 column and a refractive index detector (RID), using 0.5 mM H₂SO₄ aqueous solution as the mobile phase. The product yields (mmol/g_{cat}.L) and selectivities (%) were calculated using equations (5-6), respectively.

$${Product}_{i} Yield = \frac{{n}_{i}}{{m}_{c a t} \cdot {V}_{{H}_{2} O}} \qquad ( 5 )$$

$${Product}_{i} selectivity = \frac{{n}_{i}}{{Σ}_{i = H C O O H}^{C {H}_{3} COOH} {n}_{i}} \times 100 \qquad ( 6 )$$

nᵢ=number of moles of product
i={HCOOH, CH₃COOH}
m_{cat}=mass of catalyst
V_{H}_{₂}_{O}=volume of water

The best catalyst was also evaluated for aqueous phase conversion using CO₂, H₂ and methanol (10 mmol) as reactants and lithium iodide (10 mmol) as the co-catalyst. All other reaction conditions were identical to the above described procedure.

4.2 Aqueous Phase CO₂ Conversion using Co-catalysts

Liquid phase CO₂ hydrogenation reactions were carried out in a stainless-steel autoclave stirred batch reactor. The reaction mixture consisted of 0.4 g of the reduced catalyst (ZIF-67-T), 0.8 g NaOH and 40 mL Milli-Q water. The sealed reactor was first purged with H₂ to eliminate air from the vapor space and then pressurised with the desired amounts of H₂ and CO₂. The reactor was then heated up to 250° C. under continuous stirring at 200 rpm. Liquid samples were collected at reaction times of Oh (when the desired temperature is first reached), 1 h, 2h, 4 h, 6 h and 24h. Liquid chromatography (Agilent 1220 Infinity) was used to determine product concentrations for acetic acid and formic acid. A Rezex RHM-Monosaccharide column (60° C. column temperature) was used with a refractive index detector (40° C.) and 0.5 mM H₂SO₄ (aq) mobile phase (0.600 ml/min) for the detection of these organic acids.

### 5. Catalyst Activities

5.1 Role of Fe based zeolite and MOF catalysts

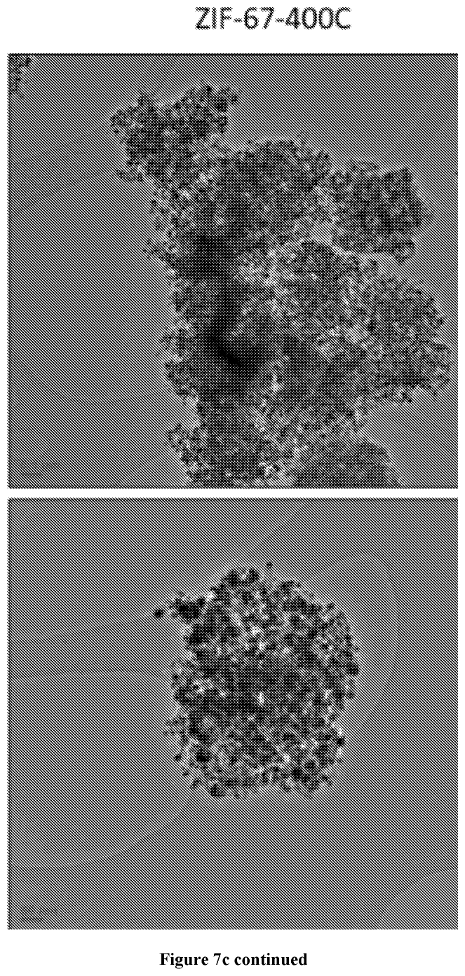

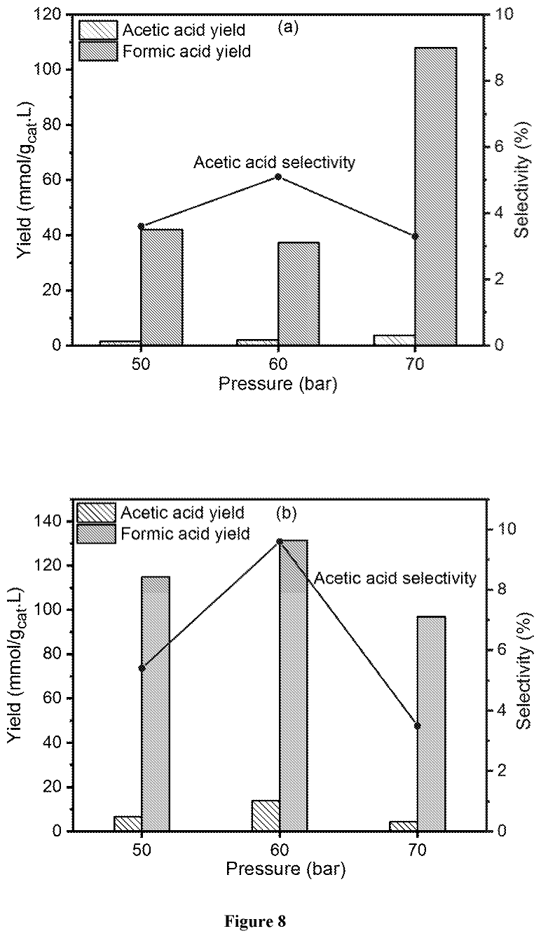

All catalysts showed some activity for AA production; however, T-MIL-88B was the most active and selective catalyst with a best yield of 504 mmol/g_{cat}.L and AA selectivity of 92.4%. Both Fe/CBEA and T-Fe/MIL-101 provide lower activity for CO₂ hydrogenation and >90% selectivity for FA production (FIGS. 8a-b). With increasing pressure, the yield increased initially but the AA selectivity peaked at 60 bar for both Fe/CBEA and T-Fe/MIL-101. However, the AA yield and selectivity increases with increasing pressure for T-MIL-88B (FIG. 8c). Since Fe was present in the structural framework of T-MIL-88B, the degraded catalyst likely consists of encapsulated active metal sites dispersed evenly in a carbon matrix. The high AA activity and the selectivity over T-MIL-88B catalyst is most likely due to the presence of both Fe0 and Fe₃O₄ which assist the hydrogenation and C—C coupling reactions, respectively.

5.2 Extent of Reaction with Time

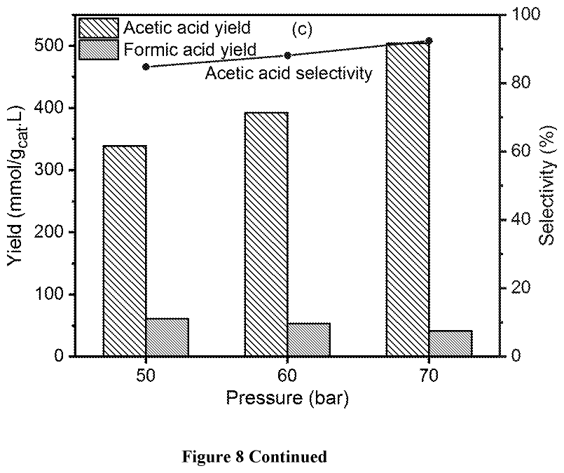

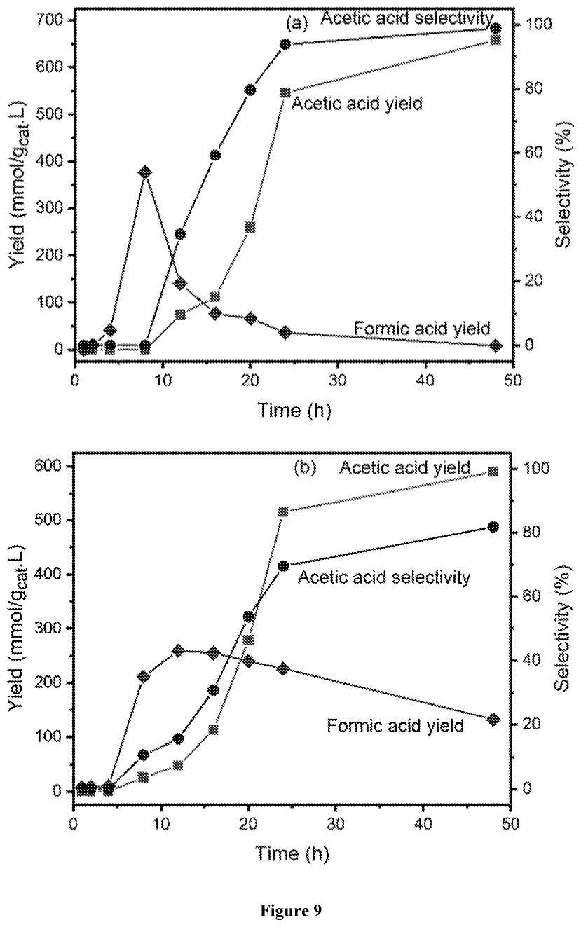

FIG. 9a illustrates the extent of reaction over T-MIL-88B to produce AA and FA via CO₂ hydrogenation with CH₃I as the starting material in the aqueous media. The reaction proceeds via formation of FA as the initial product, whereas AA was not detected until after 8h of reaction. The AA yield and selectively sharply increased between 12 to 24 h, thereafter gradually increasing to 657.6 mmol/g_{cat}.L and 98.8%, respectively, at 48h as the reaction approached equilibrium conversion. Based on the initial CH₃I concentration (10 mmol), 100% conversion and 100% selectivity for AA was achieved, within the range of measurement errors. However, as discussed later, CO₂ first converts into FA and after reaching the maximum yield (377.4 mmol/g_{cat}. L) at 8 h, the FA yield decreases sharply until the end of reaction at 48 h when the FA yield was measured at 8.1 mmol/g_{cat}. L. Though, since CH₃I is consumed by this time, the residual FA cannot convert into AA. Therefore, CO₂ hydrogenated into carboxylic acids occurs with an AA selectivity of 98.8%.

When CH₃OH (10 mmol) was used as a reactant with Lil as a co-catalyst (FIG. 9b), in otherwise identical reaction conditions, the reaction generates in situ CH₃I and hence the peak of FA is broader than FIG. 9a. The AA yield and selectivity increased more gradually and achieved a comparative but lower yield of 590.1 mmol/g_{cat}.L at 81.7% selectivity after 48 h

### 5.3 Catalyst Recycling Study and Reusability

The catalyst recyclability was investigated using CO₂, H₂ and CH₃OH (10 mmol) as reactants and lithium iodide (10 mmol) as the co-catalyst at 150° C., equimolar H₂/CO₂ with 70 bar pressure at room temperature and 200 RPM stirring speed. After each cycle, the catalyst was recovered from the product mixture via centrifugation at 8500 RPM for 1 h and without any treatment, resuspended into a fresh reaction mixture at the same initial conditions. After five cycles, the centrifuged catalyst was dried overnight in oven at 70° C. and stored in air tight glass vial for its characterisation.

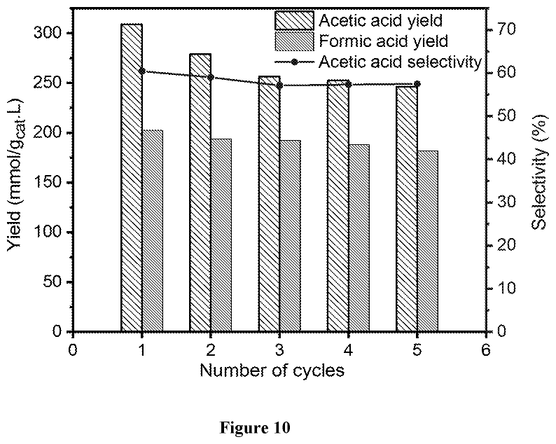

FIG. 10 shows that the catalyst was able to be recycled in the process. The PXRD of the spent catalyst after five cycles (FIG. 1c), TEM image (FIG. 3f) and PSD (FIG. 3h) of spent catalyst after 48 h confirmed that the structure is stable and there was no sintering or agglomeration of Fe and Fe₃O₄ nanoparticles in T-MIL-88B.

### 5.4 Catalytic Activity of ZIF-67-T

As shown in FIG. 11a, product yields varied significantly with the catalyst reduction temperature and the highest yield of 27.6 mmol/L/g_{cat} was observed for ZIF-67-375C after 24h reaction time. The selectivity of acetic acid was determined at the end of the reaction at t=24h, with ZIF-67-375C also having the highest selectivity of 61% as shown in FIG. 11b. Acetic acid and formic acid were the main products detected by HPLC analysis and selectivity was calculated as follows:

$$Selectivity = \frac{Yield ( AA )}{Yield ( AA ) + Yield ( F A )}$$

The reaction profile for ZIF-67-375C indicates that formic acid was quickly being converted to acetic acid very early in the reaction, which limited the peak formic acid yield. The formic acid yield peaked at around 26 mmol/L/g_{cat} which was significantly lower than the peak formic acid concentrations seen for ZIF-67-300C (80.8 mmol/L/g_{cat}) and ZIF-67-400C (218 mmol/L/g_{cat}). This highlights the greater ability of the ZIF-67-375C catalyst to convert the formic acid intermediate into acetic acid, which can be attributed to its excellent dispersion of active cobalt nanoparticles. The XRD results for ZIF-67-300C did not show any reduction of cobalt ions to the metallic cobalt phase and lack of this active phase may explain why there was very little C—C coupling to convert formic acid to acetic acid. The active metallic cobalt phase was present in the ZIF-67-400C catalyst however, TEM results show that the amorphous nature of the catalyst resulted in agglomerated particles and signs of cobalt starting to aggregate into larger clusters. Both these factors can lead to reduced catalytic activity.

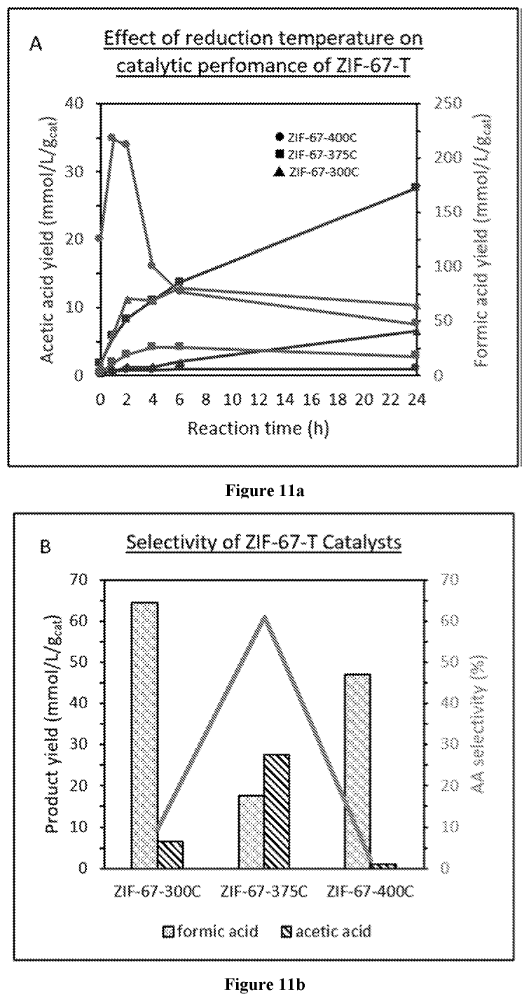

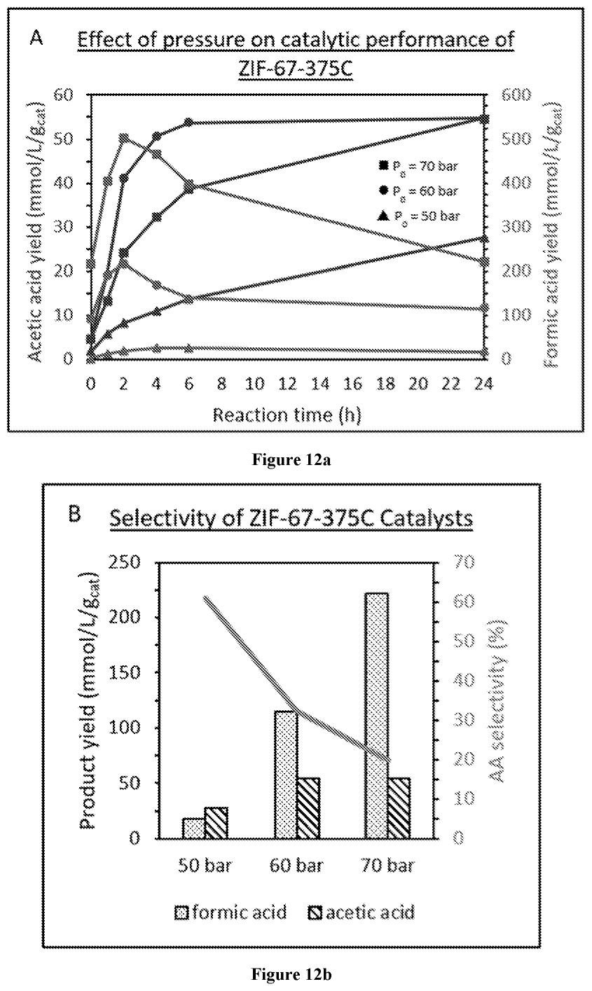

The effect of reaction pressure was also investigated for the best performed thermally transformed ZIF-67 catalyst (ZIF-67-375C). The reaction was pressurised initially to 50, 60 and 70 bar, while keeping all other reaction parameters constant, and FIG. 12a shows the concentrations of formic acid and acetic acid over time. As expected, the formic acid yield increased with higher initial reaction pressure. Starting with higher initial partial pressures of H₂ and CO₂ would lead to greater availability of these dissolved gases in the liquid phase, which is where CO₂ hydrogenation to formic acid can take place. Increasing the starting pressure from 50 to 60 bar resulted in an increase in acetic acid yield owing to an increase in the available formic acid intermediate. A further increase in pressure to 70 bar, however, resulted in a similar acetic yield of 55 mmol/L/g_{cat} after 24h reaction time. With more formic acid being produced at 70 bar, the reaction had not yet reached equilibrium after 24h as the acetic acid yield was still increasing at this point. Running these experiments for longer reaction times is required to see the peak concentration of acetic acid.

### 6. Reaction Mechanism Investigation Using T-MIL-88B

The reaction mechanism was explored by designing two different experiments. In the first reaction system, FA and CH₃I were used as reactants and the experiment was conducted in water by using T-MIL-88B as catalyst at 150° C. under 35 bar hydrogen and 200 RPM stirring speed. Typically, 40 mL H₂O, 0.4 g of T-MIL-88B, 5 mmol (312.5 mmol/g_{cat}.L) of HCOOH and 10 mmol (625 mmol/g_{cat}.L) of CH₃I were added in Teflon-liner and reactor was sealed. After achieving the above described conditions, 2 mL liquid sample was withdrawn from the reactor after 1 h of reaction through dip tube valve. Subsequently, reactor was re-pressurized again with hydrogen to maintain the pressure and several samples were collected for HPLC analysis at different reaction times (2, 4, 8, 12 and 24) by adopting this method.

In the second reaction system, aqueous phase CO₂ hydrogenation with CH₃OH (10 mmol) and LiI (10 mmol) was performed over MIL-88B (0.4 g) for 48 h at 150° C., 40 mL H₂O, equimolar H₂/CO₂ under 70 bar at room temperature and 200 RPM stirring speed. After 48 h, the reactor was cooled to room temperature. Both liquid and gas samples were collected for product analysis, where, gas sample was analysed through Shimadzu 2014 GC coupled with TCD and FID detectors, respectively.

### 6.1 Proposed Reaction Pathway

The reaction mechanism of the hydrocarboxylation of CH₃OH in an organic solvent proceeds via reaction of CH₃OH with Lil to produce CH₃I and LiOH which is similar to the carbonylation of methanol (Monsanto processes) followed by formation of CH₃Rh*I due to the insertion of CH₃I into a Rh* complexing catalyst (Qian et al). Further, CO₂ is inserted into CH₃—Rh bond to produce CH₃COORh*I. Finally, CH₃COOH is formed via reduction of CH₃COORh*I with H₂ molecule in the presence of Ru* to produce HI as an intermediate. Whereas, Lil is regenerated in-situ via HI formation which reacts with LiOH to produce H₂O and LiI. However, here we show aqueous phase methanol hydrocarboxylation in which the reaction pathway deviates from the published works and FA is formed as an intermediate.

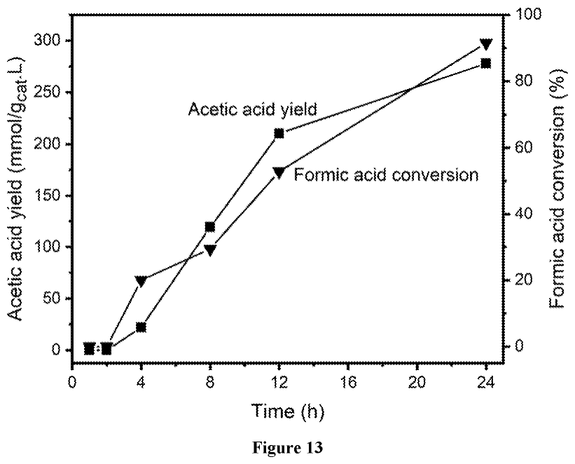

We demonstrate that FA can react with CH₃I in water over T-MIL-88B in H₂ atmosphere (FIG. 13). The conversion of FA closely follows AA yield and after 24 h of the reaction FA conversion of 91.5% is achieved with 100% AA selectivity.

Next, we demonstrate the aqueous phase hydrocarboxylation of CH₃OH using T-MIL-88B as catalyst and Lil as co-catalyst. Here both liquid and gas samples were collected after 48 of reaction. The liquid sample showed only the presence of HCOOH and CH₃COOH with 81.7% acetic acid selectivity (FIG. 9b).

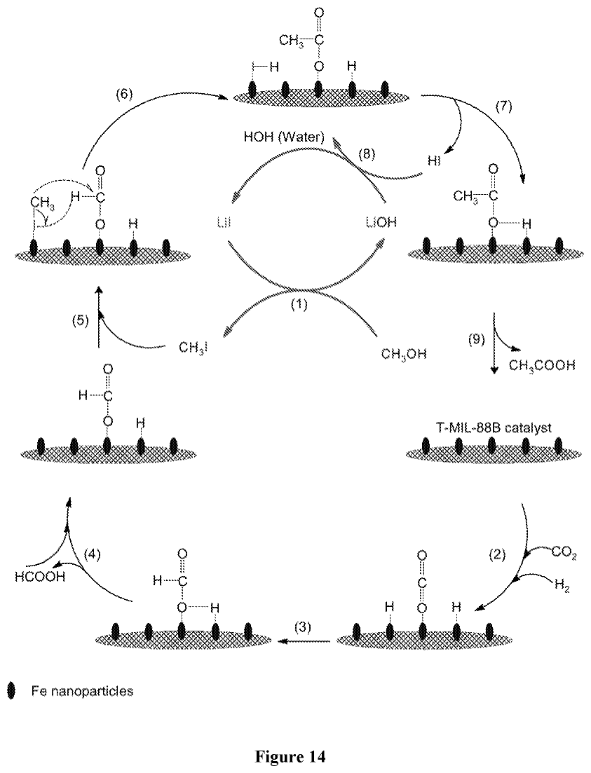

FIG. 14 shows the proposed reaction pathway for acetic acid production via hydrocarboxylation of CH₃OH over T-MIL-88B. CO₂ and H₂ adsorb over the catalyst and convert into FA, which may desorb. Subsequently, the adsorbed formate species reacts with iodomethane (CH₃I) to allow C—C a coupling reaction to take place which generates an acetate species and HI as the by-product. Finally, the acetate species is converted into acetic acid, whilst Lil might be regenerated from LiOH and HI (step 8).

Without wishing to be bound by theory, it is understood that CO₂ hydrogenation to acetic acid in aqueous media using a thermally transformed cobalt catalyst proceeds through the generation of HCOOH and *CH₂ as reaction intermediates, which are then able to undergo C—C coupling on the CoO/Co interfaces of the catalyst.

### 7. Conclusion

The Examples describe the design and synthesis of thermally decomposed Fe- and Co-containing metal organic framework-based catalysts, which exhibit high catalytic proficiency for aqueous phase CO₂ transformation into acetic acid.

The catalytic activity and the structural property of the aforementioned Fe-containing catalyst has been also compared with synthesized Fe/CBEA and T-Fe/MIL-101. Regarding the Fe-containing catalysts, PXRD and XPS analyses confirmed that T-MIL-88B consisted with both metallic Fe⁰ and Fe₃O₄ phase which are likely to catalyse hydrogenation and C—C coupling reactions directed to the generation of acetic acid via CO₂ hydrogenation. T-MIL-88B showed higher catalytic activity as compared to Fe/CBEA and T-Fe/MIL-101 during aqueous phase CO₂ hydrogenation with CH₃I additive under identical reaction conditions. The experimental data thus supports that catalysts having both Fe (0) sites and Fe (II) (III) sites, are useful in catalysing the production of acetic acid.

In the presence of CH₃OH and Lil additive, the maximum 590.1 mmol/g_{cat}.L of AA yield with 81.7% selectivity was achieved over T-MIL-88B after 48 h at 150° C., equimolar H₂/CO₂ under 70 bar at room temperature and 200 RPM stirring speed.

The time on stream study also proposed that the acetic acid maybe produced through a formic acid intermediate. The controlled reaction study (formic acid as reactant) also provided further evidence that formic acid is converted into acetic acid in presence of iodomethane in water.

The plausible reaction pathway proposed that CO₂ first converted into formic acid through hydrogenation followed by its transformation into acetic acid. Accordingly, by controlling reaction parameters such as reaction time and/or omitting reactants such as methyl halide (or methanol and metal halide), the process can be used to produce formic acid.

The catalyst recycling study demonstrated the reusability of T-MIL-88B for five cycles of AA production via this reaction system. Subsequently, the characterization (PXRD, TEM and PSD) of used catalyst also envisioned the stability of the catalyst, with its structure being largely unchanged, and supporting further reusability of the catalyst.

Regarding the Co-containing catalyst ZIF-67-T, the catalyst demonstrated the ability to convert carbon dioxide and hydrogen into acetic acid with selectivity over production of formic acid.

### REFERENCES

Farisabadi A, Moradi M, Hajati S, Kiani M A, Espinos J P. Controlled thermolysis of MIL-101 (Fe, Cr) for synthesis of FexOy/porous carbon as negative electrode and Cr203/porous carbon as positive electrode of supercapacitor. Applied Surface Science 469, 192-203 (2019). Férey G, et al. A chromium terephthalate-based solid with unusually large pore volumes and surface area. Science 309, 2040-2042 (2005).
Guo L, Qin S, Yang B, Liang D, Qiao L. Effect of hydrogen on semiconductive properties of passive film on ferrite and austenite phases in a duplex stainless steel. Scientific reports 7, 1-6 (2017).
Hasan S, Ahmad K, Isahak W, Pudukudy M, Masdar M, Jahim J. Synthesis, Characterisation and Catalytic Activity of NiO supported Al₂O₃ for CO₂ Hydrogenation to Carboxylic Acids:Influence of Catalyst Structure. In: IOP Conference Series: Earth and Environmental Science. IOP Publishing (2019).
Hou S, et al. Green synthesis and evaluation of an iron-based metal-organic framework MIL-88B for efficient decontamination of arsenate from water. Dalton transactions 47, 2222-2231 (2018).
Koley P, et al. Leveraging Cu/CuFe204-Catalyzed Biomass-Derived Furfural Hydrodeoxygenation: A Nanoscale Metal-Organic-Framework Template Is the Prime Key. ACS applied materials & interfaces 12, 21682-21700 (2020).
Lee H, Lee W-J, Park Y—K, Ki S J, Kim B-J, Jung S—C. Liquid phase plasma synthesis of iron oxide nanoparticles on nitrogen-doped activated carbon resulting in nanocomposite for supercapacitor applications. Nanomaterials 8, 190 (2018).
Li W, et al. A short review of recent advances in CO₂ hydrogenation to hydrocarbons over heterogeneous catalysts. RSC advances 8, 7651-7669 (2018).
Li X, Zhang L, Sun Y. Titanium-Modified MIL-101 (Cr) Derived Titanium-Chromium-Oxide as Highly Efficient Oxidative Desulfurization Catalyst. Catalysts 10, 1091 (2020).
Liu J, et al. Fe-MOF-derived highly active catalysts for carbon dioxide hydrogenation to valuable hydrocarbons. Journal of CO2 Utilization 21, 100-107 (2017).
Liu Z, He W, Zhang Q, Shapour H, Bakhtari M F. Preparation of a GO/MIL-101 (Fe) Composite for the Removal of Methyl Orange from Aqueous Solution. ACS omega, (2021).
Ma Z, Song T, Yuan Y, Yang Y. Synergistic catalysis on Fe-Nx sites and Fe nanoparticles for efficient synthesis of quinolines and quinazolinones via oxidative coupling of amines and aldehydes. Chemical science 10, 10283-10289 (2019).
Ndoye B, Lebecque S, Destain J, Guiro A T, Thonart P. A new pilot plant scale acetifier designed for vinegar production in Sub-Saharan Africa. Process Biochemistry 42, 1561-1565 (2007).
Nirumand L, Farhadi S, Zabardasti A, Khataee A. Synthesis and sonocatalytic performance of a ternary magnetic MIL-101 (Cr)/RGO/ZnFe204 nanocomposite for degradation of dye pollutants. Ultrasonics sonochemistry 42, 647-658 (2018).
Pal P, Nayak J. Acetic acid production and purification: critical review towards process intensification. Separation & Purification Reviews 46, 44-61 (2017).
Qian Q, Zhang J, Cui M, Han B. Synthesis of acetic acid via methanol hydrocarboxylation with CO 2 and H 2. Nature communications 7, 1-7 (2016)
Vu T A, et al. Highly photocatalytic activity of novel Fe-MIL-88B/GO nanocomposite in the degradation of reactive dye from aqueous solution. Materials Research Express 4, 035038 (2017).
Zhao M, et al. Metal-organic frameworks as selectivity regulators for hydrogenation reactions. Nature 539, 76-80 (2016).
X. Wang, Y. Yang, T. Wang, H. Zhong, J. Cheng and F. Jin, “In Situ Formed Metal Oxide/Metal Interface Enhanced C—C Coupling in CO₂ Reduction into CH₃COOH over Hexagonal Closed-Packed Cobalt”, ACS Sustainable Chemistry & Engineering, vol. 9, no. 3, pp. 1203-1212, 2021. Available: 10.1021/acssuschemeng.0c06717.
M. Potter, C. Ross, D. Gianolio, R. Rios and R. Raja, “Cobalt-containing zeolitic imidazole frameworks for C—H activation using visible-light redox photocatalysis”, Catalysis Science & Technology, vol. 10, no. 21, pp. 7262-7269, 2020. Available: 10.1039/d0cy01061h.
L. Chen et al., “Mesoporous Zeolitic Imidazolate Framework-67 Nanocrystals on Siliceous Mesocellular Foams for Capturing Radioactive Iodine”, ACS Applied Nano Materials, vol. 3, no. 6, pp. 5390-5398, 2020. Available: 10.1021/acsanm.0c00756.

1. A process for producing a compound of formula (I), or a salt thereof: 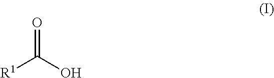
wherein R¹ is H or methyl, comprising:
a) reacting a methyl halide in the presence of hydrogen, carbon dioxide and a catalyst selected from the group consisting of i) an iron- and carbon-containing catalyst, and ii) a cobalt- and carbon-containing catalyst, to produce acetic acid and/or a salt thereof; or
b) reacting a methyl halide with carbon monoxide in the presence of a catalyst selected from the group consisting of i) an iron- and carbon-containing catalyst, and ii) a cobalt- and carbon-containing catalyst, to produce acetic acid and/or a salt thereof;
c) reacting hydrogen and carbon dioxide in the presence of a catalyst selected from the group consisting of i) an iron- and carbon-containing catalyst, and ii) a cobalt- and carbon-containing catalyst, to produce formic acid and/or a salt thereof; or
d) reacting hydrogen and carbon dioxide in the presence of a cobalt- and carbon-containing catalyst to produce acetic acid and/or a salt thereof;
wherein the iron- and carbon-containing catalyst comprises Fe (0) sites and Fe (II)/(III) sites; and wherein the cobalt- and carbon-containing catalyst comprises Co (0) sites and Co (II) sites.

2. The process for producing a compound of formula (I), or a salt thereof, as claimed in claim 1: 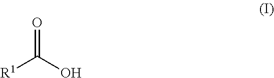
wherein R¹ is H or methyl, comprising:
a) reacting a methyl halide in the presence of hydrogen, carbon dioxide and an iron- and carbon-containing catalyst to produce acetic acid and/or a salt thereof; or
b) reacting a methyl halide with carbon monoxide in the presence of an iron- and carbon-containing catalyst to produce acetic acid and/or a salt thereof; or
c) reacting hydrogen and carbon dioxide in the presence of an iron- and carbon-containing catalyst, to produce formic acid and/or a salt thereof;
wherein the iron- and carbon-containing catalyst comprises Fe (0) sites and Fe (II)/(III) sites.

3. The process according to claim 2, wherein the catalyst comprises iron-containing particles having a mean equivalent particle diameter of up to 9.8 nm, and/or a mean particle long axis of up to 9.8 nm.

4. The process according to claim 2 or 3, wherein the catalyst has more than 45 wt % iron, and has a hydrogen content in the range of from 0.4 to 0.8 wt %.

5. The process according to any of claims 2 to 4, wherein the catalyst has an S_{BET} value of at least 150 m² g⁻¹.

6. The process according to any of claims 2 to 5, wherein the catalyst is a thermally-treated iron-containing metal-organic framework (MOF), which has been thermally treated under a hydrogen-containing atmosphere.

7. The process according to claim 6, wherein the iron-containing MOF has been thermally treated under a hydrogen/argon atmosphere.

8. The process according to claim 6 or 7, wherein the iron-containing MOF has been thermally treated at a temperature in the range of from 400 to 600° C.

9. The process according to any of claims 6 to 8, wherein the iron-containing MOF has been thermally treated at about 500° C.

10. The process according to any of claims 2 to 9, wherein the catalyst comprises Fe (0) sites and Fe₃O₄ sites.

11. The process according to any of claims 2 to 10, wherein the wt % of iron in the catalyst is in the range of from 30 to 70 wt %.

12. The process according to claim 11, wherein the wt % of iron in the catalyst is in the range of from 45 to 55 wt %.

13. The process according to any of claims 2 to 12, wherein the wt % of sodium in the catalyst is in the range of from 15 to 25 wt %.

14. The process according to any of claims 2 to 13, wherein wt % of carbon in the catalyst is in the range of from 10 to 20 wt %.

15. The process according to any of claims 2 to 14, wherein the catalyst is thermally treated MIL-88B, and wherein the MIL-88B has been thermally treated under a hydrogen/argon atmosphere at a temperature in the range of from 450 to 550° C. for a time period in the range of from 4 to 6 hours.

16. The process for producing a compound of formula (I), or a salt thereof, as claimed in claim 1: 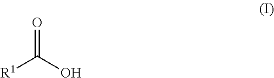
wherein R¹ is H or methyl, comprising:
e) reacting a methyl halide in the presence of hydrogen, carbon dioxide and a cobalt- and carbon-containing catalyst to produce acetic acid and/or a salt thereof; or
f) reacting a methyl halide with carbon monoxide in the presence of a cobalt- and carbon-containing catalyst, to produce acetic acid and/or a salt thereof;
g) reacting hydrogen and carbon dioxide in the presence of a cobalt- and carbon-containing catalyst to produce formic acid and/or a salt thereof; or
h) reacting hydrogen and carbon dioxide in the presence of a cobalt- and carbon-containing catalyst to produce acetic acid and/or a salt thereof;
wherein the cobalt- and carbon-containing catalyst comprises Co (0) sites and Co (II) sites.

17. The process according to claim 16, wherein the catalyst is a thermally-treated cobalt-containing metal-organic framework (MOF), which has been thermally treated under a hydrogen-containing atmosphere.

18. The process according to claim 17, wherein the cobalt-containing MOF has been thermally treated under a hydrogen/argon atmosphere.

19. The process according to claim 17 or 18, wherein the cobalt-containing MOF has been thermally treated at a temperature in the range of from 360 to 390° C.

20. The process according to any of claims 17 to 19, wherein the cobalt-containing MOF has been thermally treated at about 375° C.

21. The process according to any of claims 17 to 20, wherein the catalyst comprises Co (0) having a face centred cubic (FCC) structure.

22. The process according to any of claims 16 to 21, wherein the catalyst is thermally treated ZIF-67, and wherein the ZIF-67 has been thermally treated under a hydrogen/argon atmosphere at a temperature in the range of about 360 to 390° C. for a time period in the range of from 3 to 5 hours.

23. The process according to any of claims 1 to 15, wherein the process comprises reacting a methyl halide in the presence of hydrogen, carbon dioxide and an iron- and carbon-containing catalyst to produce acetic acid and/or a salt thereof.

24. The process according to any of claims 1 to 15, wherein the process comprises reacting a methyl halide with carbon monoxide in the presence of an iron- and carbon-containing catalyst to produce acetic acid and/or a salt thereof.

25. The process according to any of claims 1 and 16 to 22, wherein the process comprises reacting hydrogen and carbon dioxide in the presence of a cobalt- and carbon-containing catalyst to produce acetic acid and/or a salt thereof.

26. The process according to any of claims 1 to 15, wherein the process comprises reacting hydrogen and carbon dioxide in the presence of an iron- and carbon-containing catalyst, to produce formic acid and/or a salt thereof.

27. The process according to any of claims 1 to 24, wherein the process comprises step a) or step b), and the methyl halide is methyl iodide.

28. The process according to any of claim 1 to 23 or 27, wherein the process comprises step a) and the process comprises reacting methanol with a metal halide to produce the methyl halide.

29. The process according to any of claims 1 to 22, 24, or 27 to 28, wherein the process comprises step b) and the process comprises reacting methanol with a metal halide and/or HI to produce the methyl halide.

30. The process according to claim 28 or 29, wherein the metal halide is a metal iodide and the methyl halide is methyl iodide.

31. The process according to claim 30, wherein the metal halide is lithium iodide.

32. The process according to any of claims 1 to 23, 27 to 28 or 30 to 31, wherein the process comprises step a) and the reaction to produce acetic acid and/or a salt thereof is carried out under a pressurised hydrogen and carbon dioxide atmosphere.

33. The process according to claim 32, wherein the bar ratio of hydrogen to carbon dioxide is about 1:1.

34. The process according to any of claims 1 to 23, 27 to 28, or 30 to 33, wherein the process comprises step a) and the reaction to produce acetic acid and/or a salt thereof is conducted at a pressure in the range of from 50 to 100 bar.

35. The process according to claim 34, wherein the reaction to produce acetic acid and/or a salt thereof is conducted at a pressure of about 70 bar.

36. The process according to any of claims 1 to 23, 27 to 28, or 30 to 35, wherein the process comprises step a) and the reaction to produce acetic acid and/or a salt thereof is conducted under aqueous conditions.

37. The process according to any of claims 1 to 23, 27 to 28, or 30 to 36, wherein the process comprises step a) and the reaction to produce acetic acid and/or a salt thereof is carried out with stirring and/or agitation of the reaction mixture.

38. The process according to any of claims 28 or 30 to 37, wherein the process comprises step a) and the reaction to product acetic acid and/or a salt thereof comprises stirring a mixture of methanol, metal halide and water in the presence of the solid catalyst under a hydrogen and carbon dioxide atmosphere.

39. The process according to any of claims 1 to 24, or 27 to 38, wherein the reaction to produce acetic acid and/or a salt thereof is carried out at a temperature in the range of from 75° C. to 200° C.

40. The process according to claim 39, wherein the reaction to produce acetic acid and/or a salt thereof is carried out at about 150° C.

41. The process according to any of claims 1 to 23, 27 to 28, or 30 to 40, wherein the process comprises step a) and the reacting step is carried out for a time period in the range of from 12 to 48 hours.

42. The process according to any of claims 1 to 24 or 27 to 41, wherein the process comprises step a) or step b) and, following the reaction to produce acetic acid and/or a salt thereof, the catalyst is recovered and recycled to the process.

43. The process according to any of claim 1, 16 to 22, or 25, wherein the process comprises step d) and the reacting step is carried out for a time period in the range of from 12 to 72 hours.

44. The process according to any of claim 1, 16 to 22, 25, or 43, wherein the process comprises step d), and the reaction to produce acetic acid and/or a salt thereof is conducted at a pressure in the range of from 30 to 100 bar.

45. The process according to any of claims 1, 16 to 22, 25, or 43 to 44, wherein the process comprises step d), and the reaction to produce acetic acid and/or a salt thereof is conducted at a temperature in the range of from 150 to 350° C.

46. The process according to any of claims 1, 16 to 22, 25, or 43 to 45, wherein the bar ratio of hydrogen to carbon dioxide is in the range of from 2:1 to 3:1.

47. The process according to any of claims 1, 16 to 22, 25, or 43 to 46, wherein the reaction to produce acetic acid and/or a salt thereof is carried out in the presence of a base.

48. A process for producing a product selected from the group consisting of an acetate ester, an ether acetate, a metal acetate, acetic anhydride, acrylic acid or an acrylate, comprising producing acetic acid and/or a salt thereof by a process comprising step a), step b) or step d) as defined in any of claims 1 to 25 or 27 to 47, and converting the acetic acid and/or salt thereof into an acetate ester, an ether acetate, a metal acetate, acetic anhydride, acrylic acid or an acrylate.

49. The process according to claim 48, wherein the product is selected from the group consisting of polyvinyl acetate, vinyl acetate, a metal acetate, a cellulose acetate, ethyl acetate, n-butyl acetate, isobutyl acetate, propyl acetate, ethylene glycol monoethyl ether acetate (EEA), ethylene glycol monobutyl ether acetate (EBA), and propylene glycol monomethyl ether acetate (PMA or PGMEA), acetic anhydride, acrylic acid and/or acrylate.

50. An iron- and carbon-containing catalyst, wherein the catalyst comprises Fe (0) sites and Fe (II)/Fe (III) sites, and wherein:
e) the iron particles have a mean equivalent particle diameter of up to 9.8 nm, and/or a mean particle long axis of up to 9.8 nm; and/or
f) the catalyst has more than 45 wt % iron, and has a hydrogen content in the range of from 0.4 to 0.8 wt %; and/or
g) the catalyst comprises an S_{BET} value of at least 150 m² g⁻¹; and/or
h) the catalyst is a thermally treated iron-containing metal-organic framework (MOF), which has been thermally treated under a hydrogen-containing atmosphere.

51. The iron- and carbon-containing catalyst according to claim 50, wherein at least 90% of the iron particles have an equivalent particle diameter in the range of from 3 to 20 nm, and/or at least 90% of the iron particles have a particle long axis in the range of from 3 to 20 nm.

52. The iron- and carbon-containing catalyst according to claim 50 or 51, wherein the ratio of Fe (0) sites to Fe (II)/(III) sites is in the range of from 5:1 to 1:5

53. A cobalt- and carbon-containing catalyst, wherein the catalyst comprises Co (0) sites and Co (II) sites.

54. The cobalt- and carbon-containing catalyst according to claim 53, wherein the catalyst is a thermally-treated cobalt-containing metal-organic framework (MOF), which has been thermally treated under a hydrogen-containing atmosphere.

55. The cobalt- and carbon-containing catalyst according to claim 54, wherein the cobalt-containing MOF has been thermally treated under a hydrogen/argon atmosphere.

56. The cobalt- and carbon-containing catalyst according to claim 54 or 55, wherein the cobalt-containing MOF has been thermally treated at about 375° C.

57. The cobalt- and carbon-containing catalyst according to any of claims 53 to 56, wherein the catalyst comprises Co (0) having a face centred cubic (FCC) structure.

58. The cobalt- and carbon-containing catalyst according to any of claims 53 to 57, wherein the catalyst is thermally treated ZIF-67, and wherein the ZIF-67 has been thermally treated under a hydrogen/argon atmosphere at a temperature in the range of about 360 to 390° C. for a 5 time period in the range of from 3 to 5 hours.
